SEQUENCE LISTING (45 sequences)

SEQ ID NO: 1 - DNA, 6742 bp, Artificial Sequence
  M281-LC expression plasmid
  promoter (47)..(726): CMV Promoter
  CDS (754)..(1467): M281_LC
  misc_feature (1514)..(2111): WRPE
  misc_feature (2115)..(2386): TK pA signal
  promoter (2855)..(3224): SV40 early promoter
  misc_feature (3260)..(4054): Neo-R
  polyA_signal (4230)..(4360): SV40 pA signal
  rep_origin (4743)..(5416): pUC ori
  misc_feature (5561)..(6421): Amp-R
  promoter (6422)..(6520): bla promoter
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttggatccgccaccatggaaaccgatacactgctgctgtgggtgctgttgctctgggtgccaggatctacaggccagtctgctctgactcagcctgcctctgtttctggctcccctggccagtctatcaccatctcttgtaccggcaccggctccgacgtgggctcttataacctggtgtcctggtatcagcagcaccccggaaaggcccctaagctgatgatctacggcgactctgaacggccttccggcgtgtccaatagattctccggctccaagtccggcaataccgcctctctgacaatcagtggactgcaggccgaggacgaggccgactactactgttcttcttacgccggctccggcatctacgtgttcggcaccggaacaaaagtgaccgtgctgggacagcctaaggccgctccttctgtgaccctgtttcctccatcctccgaggaactgcaggctaacaaggctaccctcgtgtgcctgatctccgatttttaccctggcgctgtgaccgtggcctggaaggctgatagttctcctgtgaaggccggcgtggaaaccaccacaccttccaagcagtccaacaacaaatacgccgcctcctcctacctgtctctgacccctgaacagtggaagtcccacaagtcctacagctgccaagtgacccatgagggctccaccgtggaaaagacagtggcccctaccgagtgctcctgatgactcgagaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 2 - protein, 236 aa, Artificial Sequence
  Synthetic Construct
METDTLLLWVLLLWVPGSTGQSALTQPASVSGSPGQSITISCTGTGSDVGSYNLVSWYQQHPGKAPKLMIYGDSERPSGVSNRFSGSKSGNTASLTISGLQAEDEADYYCSSYAGSGIYVFGTGTKVTVLGQPKAAPSVTLFPPSSEELQANKATLVCLISDFYPGAVTVAWKADSSPVKAGVETTTPSKQSNNKYAASSYLSLTPEQWKSHKSYSCQVTHEGSTVEKTVAPTECS

SEQ ID NO: 3 - DNA, 7429 bp, Artificial Sequence
  M281-HC expression plasmid
  promoter (47)..(726): CMV promoter
  CDS (754)..(2154): M281_HC
  misc_feature (2201)..(2798): WRPE
  misc_feature (2802)..(3073): TK pA signal
  promoter (3542)..(3911): SV40 ealry promoter
  misc_feature (3947)..(4741): Neo-R
  polyA_signal (4917)..(5047): SV40 pA signal
  rep_origin (5430)..(6103): pUC ori
  misc_feature (6248)..(7108): Amp-R
  promoter (7109)..(7207): bla promoter
gttaggcgttttgcgctgcttcgcgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccggactctagaggatcgaacccttggatccgccaccatggaaaccgatacactgctgctgtgggtgctgttgctctgggttccaggatctacaggcgaggtgcagctgttggaatctggcggaggattggttcagcctggcggctctctgagactgtcttgtgctgcctctggcttcaccttctctacctacgctatgggctgggtccgacaggctcctggaaaaggactggaatgggtgtcctctatcggcgcctctggcagccagaccagatacgctgattccgtgaagggcagattcaccatcagccgggacaactccaagaacaccctgtacctgcagatgaactccctgagagccgaggacaccgccgtgtactattgtgctagactggccatcggcgactcttattggggccagggaacaatggtcaccgtgtcctctgcttccaccaagggaccttccgtgtttcctctggctccttccagcaagtctacctctggtggaaccgctgctctgggctgcctggtcaaggattactttcctgagcctgtgaccgtgtcttggaactccggtgctctgacatccggcgtgcacacatttccagctgtgctgcagtcctccggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtcctccatgtcctgctccagaactgctcggcggaccctctgtgttcctgtttccacctaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacgcctccacctacagagtggtgtctgtgctgaccgtgctgcaccaggattggctgaacggcaaagagtacaagtgcaaggtgtccaacaaggccctgcctgctcctatcgaaaagaccatctccaaggctaagggccagcctcgggaacctcaggtttacaccctgcctccatctcgggaagagatgaccaagaaccaggtgtccctgacctgcctcgtgaagggattctacccttccgatatcgccgtggaatgggagtctaacggccagccagagaacaactacaagacaacccctcctgtgctggactccgacggctcattcttcctgtactccaagctgacagtggacaagtctcggtggcagcagggcaacgtgttctcctgttctgtgatgcacgaggccctgcacaaccactacacacagaagtccctgtctctgtcccctggctgatgactcgagaagggttcgatccctaccggttagtaatgagtttgatatctcgacaatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcctggaacgggggaggctaactgaaacacggaaggagacaataccggaaggaacccgcgctatgacggcaataaaaagacagaataaaacgcacgggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcagatctgcgcagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaggacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgcgcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgagcgggactctggggttcgcgaaatgaccgaccaagcgacgcccaacctgccatcacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcgacggatcgggagatctcccgatcccctatggtcgactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagg

SEQ ID NO: 4 - protein, 465 aa, Artificial Sequence
  Synthetic Construct
METDTLLLWVLLLWVPGSTGEVQLLESGGGLVQPGGSLRLSCAASGFTFSTYAMGWVRQAPGKGLEWVSSIGASGSQTRYADSVKGRFTISRDNSKNTLYLQMNSLRAEDTAVYYCARLAIGDSYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 5 - DNA, 720 bp, Artificial Sequence
  M281_LC nucleic acid sequence
atggaaaccgatacactgctgctgtgggtgctgttgctctgggtgccaggatctacaggccagtctgctctgactcagcctgcctctgtttctggctcccctggccagtctatcaccatctcttgtaccggcaccggctccgacgtgggctcttataacctggtgtcctggtatcagcagcaccccggaaaggcccctaagctgatgatctacggcgactctgaacggccttccggcgtgtccaatagattctccggctccaagtccggcaataccgcctctctgacaatcagtggactgcaggccgaggacgaggccgactactactgttcttcttacgccggctccggcatctacgtgttcggcaccggaacaaaagtgaccgtgctgggacagcctaaggccgctccttctgtgaccctgtttcctccatcctccgaggaactgcaggctaacaaggctaccctcgtgtgcctgatctccgatttttaccctggcgctgtgaccgtggcctggaaggctgatagttctcctgtgaaggccggcgtggaaaccaccacaccttccaagcagtccaacaacaaatacgccgcctcctcctacctgtctctgacccctgaacagtggaagtcccacaagtcctacagctgccaagtgacccatgagggctccaccgtggaaaagacagtggcccctaccgagtgctcctgatgactcgag

SEQ ID NO: 6 - DNA, 1407 bp, Artificial Sequence
  M281_HC nucleic acid sequence
atggaaaccgatacactgctgctgtgggtgctgttgctctgggttccaggatctacaggcgaggtgcagctgttggaatctggcggaggattggttcagcctggcggctctctgagactgtcttgtgctgcctctggcttcaccttctctacctacgctatgggctgggtccgacaggctcctggaaaaggactggaatgggtgtcctctatcggcgcctctggcagccagaccagatacgctgattccgtgaagggcagattcaccatcagccgggacaactccaagaacaccctgtacctgcagatgaactccctgagagccgaggacaccgccgtgtactattgtgctagactggccatcggcgactcttattggggccagggaacaatggtcaccgtgtcctctgcttccaccaagggaccttccgtgtttcctctggctccttccagcaagtctacctctggtggaaccgctgctctgggctgcctggtcaaggattactttcctgagcctgtgaccgtgtcttggaactccggtgctctgacatccggcgtgcacacatttccagctgtgctgcagtcctccggcctgtactctctgtcctctgtcgtgaccgtgccttctagctctctgggcacccagacctacatctgcaacgtgaaccacaagccttccaacaccaaggtggacaagaaggtggaacccaagtcctgcgacaagacccacacctgtcctccatgtcctgctccagaactgctcggcggaccctctgtgttcctgtttccacctaagcctaaggacaccctgatgatctctcggacccctgaagtgacctgcgtggtggtggatgtgtctcacgaggacccagaagtgaagttcaattggtacgtggacggcgtggaagtgcacaacgccaagaccaagcctagagaggaacagtacgcctccacctacagagtggtgtctgtgctgaccgtgctgcaccaggattggctgaacggcaaagagtacaagtgcaaggtgtccaacaaggccctgcctgctcctatcgaaaagaccatctccaaggctaagggccagcctcgggaacctcaggtttacaccctgcctccatctcgggaagagatgaccaagaaccaggtgtccctgacctgcctcgtgaagggattctacccttccgatatcgccgtggaatgggagtctaacggccagccagagaacaactacaagacaacccctcctgtgctggactccgacggctcattcttcctgtactccaagctgacagtggacaagtctcggtggcagcagggcaacgtgttctcctgttctgtgatgcacgaggccctgcacaaccactacacacagaagtccctgtctctgtcccctggctgatgactcgag

SEQ ID NO: 7 - protein, 236 aa, Artificial Sequence; identical to SEQ ID NO: 2
  M281_LC amino acid sequence

SEQ ID NO: 8 - protein, 465 aa, Artificial Sequence; identical to SEQ ID NO: 4
  M281_HC amino acid sequence

SEQ ID NO: 9 - DNA, 42 bp, Artificial Sequence
  CDR-L1 nucleic acid sequence
accggcaccggctccgacgtgggctcttataacctggtgtcc

SEQ ID NO: 10 - protein, 14 aa, Artificial Sequence
  CDR-L1 amino acid sequence
TGTGSDVGSYNLVS

SEQ ID NO: 11 - DNA, 21 bp, Artificial Sequence
  CDR-L2 nucleic acid sequence
ggcgactctgaacggccttcc

SEQ ID NO: 12 - protein, 7 aa, Artificial Sequence
  CDR-L2 amino acid sequence
GDSERPS

SEQ ID NO: 13 - DNA, 30 bp, Artificial Sequence
  CDR-L3 nucleic acid sequence
tcttcttacgccggctccggcatctacgtg

SEQ ID NO: 14 - protein, 10 aa, Artificial Sequence
  CDR-L3 amino acid sequence
SSYAGSGIYV

SEQ ID NO: 15 - DNA, 15 bp, Artificial Sequence
  CDR-H1 nucleic acid sequence
acctacgctatgggc

SEQ ID NO: 16 - protein, 5 aa, Artificial Sequence
  CDR-H1 amino acid sequence
TYAMG

SEQ ID NO: 17 - DNA, 42 bp, Artificial Sequence
  CDR-H2 nucleic acid sequence
tctatcggcgcctctggcagccagaccagatacgctgattcc

SEQ ID NO: 18 - protein, 14 aa, Artificial Sequence
  CDR-H2 amino acid sequence
SIGASGSQTRYADS

SEQ ID NO: 19 - DNA, 21 bp, Artificial Sequence
  CDR-H3 nucleic acid sequence
ctggccatcggcgactcttat

SEQ ID NO: 20 - protein, 7 aa, Artificial Sequence
  CDR-H3 amino acid sequence
LAIGDSY

SEQ ID NO: 21 - protein, 5 aa, Artificial Sequence
  CDR-H1variant 1
DYAMG

SEQ ID NO: 22 - protein, 5 aa, Artificial Sequence
  CDR-H1 variant 2
NYAMG

SEQ ID NO: 23 - protein, 14 aa, Artificial Sequence
  CDR-H2 variant 1
SIGASGAQTRYADS

SEQ ID NO: 24 - protein, 14 aa, Artificial Sequence
  CDR-H2 variant 2
SIGASGGQTRYADS

SEQ ID NO: 25 - protein, 14 aa, Artificial Sequence
  CDR-H2 variant 3
SIGSSGAQTRYADS

SEQ ID NO: 26 - protein, 58 aa, Artificial Sequence
  Z_FcRn-2
VDAKYAKEQDAAAHEIRWLPNLTFDQRVAFIHKLADDPSQSSELLSEAKKLNDSQAPK

SEQ ID NO: 27 - protein, 58 aa, Artificial Sequence
  Z_FcRn-4
VDAKYAKEWMRAAHEIRWLPNLTFDQRVAFIHKLEDDPSQSSELLSEAKKLNDSQAPK

SEQ ID NO: 28 - protein, 58 aa, Artificial Sequence
  Z_FcRn-16
VDAKYAKEFESAAHEIRWLPNLTYDQRVAFIHKLSDDPSQSSELLSEAKKLNDSQAPK

SEQ ID NO: 29 - protein, 11 aa, Artificial Sequence
  SYN746
RFTGHFGLYPC

SEQ ID NO: 30 - protein, 13 aa, Artificial Sequence
  SYN1327
RFCTGHFGGLYPC

SEQ ID NO: 31 - protein, 13 aa, Artificial Sequence
  SYN1327-modified
  MOD_RES (3)..(3): Pen
  MOD_RES (9)..(9): Sar
  MOD_RES (10)..(10): NMeLeu
RFXTGHFGXLYPC

SEQ ID NO: 32 - protein, 17 aa, Artificial Sequence
  98420-1 amino acid sequence
QRFCTGHFGGLYPCNGP

SEQ ID NO: 33 - protein, 17 aa, Artificial Sequence
  98420-2
GGGCVTGHFGGIYCNYQ

SEQ ID NO: 34 - protein, 17 aa, Artificial Sequence
  98420-3
KIICSPGHFGGMYCQGK

SEQ ID NO: 35 - protein, 17 aa, Artificial Sequence
  98420-4
PSYCIEGHIDGIYCFNA

SEQ ID NO: 36 - protein, 17 aa, Artificial Sequence
  98420-5
NSFCRGRPGHFGGCYLF

SEQ ID NO: 37 - protein, 216 aa, Artificial Sequence
  DX2504LC
QSALTQPASVSGSPGQSITISCTGTGSDVGSYNLVSWYQQHPGKAPKLMIYGDSQRPSGVSNRFSGSKSGNTASLTISGLQAEDEADYYCCSYAGSGIYVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 38 - protein, 446 aa, Artificial Sequence
  DX2504HC
EVQLLESGGGLVQPGGSLRLSCAASGFTFSEYAMGWVRQAPGKGLEWVSSIGSSGGQTKYADSVKGRFTISRDNSKNTLYLQMNSLRAEDTAVYYCARLAIGDSYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 216 aa, Artificial Sequence
  DX2507LC
QSALTQPASVSGSPGQSITISCTGTGSDVGSYNLVSWYQQHPGKAPKLMIYGDSQRPSGVSNRFSGSKSGNTASLTISGLQAEDEADYYCASYAGSGIYVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 40 - protein, 445 aa, Artificial Sequence
  DX2507HC
EVQLLESGGGLVQPGGSLRLSCAASGFTFSEYAMGWVRQAPGKGLEWVSSIGSSGGQTKYADSVKGRFTISRDNSKNTLYLQMNSLRAEDTAVYYCARLAIGDSYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 41 - protein, 10 aa, Artificial Sequence
  DX-CDR-L3
ASYAGSGIVY

SEQ ID NO: 42 - protein, 7 aa, Artificial Sequence
  DX-CDR-L2
GDSQRPS

SEQ ID NO: 43 - protein, 5 aa, Artificial Sequence
  DX-CDR-H1
EYAMG

SEQ ID NO: 44 - protein, 17 aa, Artificial Sequence
  DX-CDR-H2
SIGSSGGQTKYADSVKG

SEQ ID NO: 45 - protein, 365 aa, Artificial Sequence
  hFcRN amino acid sequence
MGVPRPQPWALGLLLFLLPGSLGAESHLSLLYHLTAVSSPAPGTPAFWVSGWLGPQQYLSYNSLRGEAEPCGAWVWENQVSWYWEKETTDLRIKEKLFLEAFKALGGKGPYTLQGLLGCELGPDNTSVPTAKFALNGEEFMNFDLKQGTWGGDWPEALAISQRWQQQDKAANKELTFLLFSCPHRLREHLERGRGNLEWKEPPSMRLKARPSSPGFSVLTCSAFSFYPPELQLRFLRNGLAAGTGQGDFGPNSDGSFHASSSLTVKSGDEHHYCCIVQHAGLAQPLRVELESPAKSSVLVVGIVIGVLLLTAAAVGGALLWRRMRSGLPAPWISLRGDDTGVLLPTPGEAQDADLKDVNVIPATA

(19) United States
(12) Patent Application Publication
(10) Pub. No.: US 2023/0220069 A1
(43) Pub. Date: Jul. 13, 2023

(54) COMPOSITIONS AND METHODS FOR TREATMENT OF GENE THERAPY PATIENTS

(71) Applicant: The Trustees of the University of Pennsylvania, Philadelphia, PA (US)

(72) Inventors: James M. Wilson, Philadelphia, PA (US); Christian Hinderer, Baltimore, MD (US); Makoto Horiuchi, Wallingford, PA (US)

(21) Appl. No.: 18/002,060

(22) PCT Filed: Jun. 16, 2021
(86) PCT No.: PCT/US2021/037575

§ 371 Date: Dec. 16, 2022

Related U.S. Application Data
(60) Provisional application No. 63/152,085, filed on Feb. 22, 2021.
(60) Provisional application No. 63/135,998, filed on Jan. 11, 2021.
(60) Provisional application No. 63/040,381, filed on Jun. 17, 2020.

(51) Int. Cl. C07K 16/28; A61K 38/21; C12N 15/86; A61K 9/00

(52) U.S. Cl. CPC C07K 16/28; A61K 38/217; C12N 15/86; A61K 9/0019; C07K 2317/622; C07K 2317/55; C07K 2317/565; C12N 2710/10041

(57) ABSTRACT

Provided herein are compositions useful for co-administering with a gene therapy vector to a patient having pre-existing neutralizing antibodies to the viral source of the gene therapy vector capsid. The compositions comprise an FcRn ligand which inhibits specific binding between FcRn and IgG.

### BACKGROUND OF THE INVENTION

Recombinant adeno-associated virus (AAV) vectors are derived from wild-type (WT) AAV which are small, non-enveloped, 4.7 kb DNA dependoviruses in the Parvoviridae family. These rAAV have demonstrated the ability to be a useful gene delivery system in a variety of tissues, including eye, liver, skeletal muscle and the central nervous system. WT AAV are highly prevalent in the human population, as they have been detected in many different human tissues [Smith-Arica J R, et al., Infection efficiency of human and mouse embryonic stem cells using adenoviral and adeno-associated viral vectors. Cloning Stem Cells 2003; 5:51-62; Friedman-Einat M, et al, Detection of adeno-associated virus type 2 sequences in the human genital tract. J Clin Microbiol 1997; 35:71-8; and Auricchio A, Rolling F. Adeno-associated viral vectors for retinal gene transfer and treatment of retinal diseases. Curr Gene Ther 2005; 5:339-48]. For example, the prevalence of natural AAV infections has been described in as much as 15% to 30% of the population (>1:20, AAV8).

While exposure to WT AAV has not been associated with any clinical pathology or disease, pre-existing neutralizing antibodies to certain AAV have been shown to exist and can prevent rAAV having the same or a serologically cross-reactive capsid can prevent tissue transduction following vector administration. For this reason, the presence of a neutralizing antibody titer greater than 1:5 is a common exclusion criteria for AAV-based systemic gene therapy. H C Verdera, et al, Molecular Therapy, Vol. 28, No. 3, pp 723-746 (March 2020). NAb titer higher than 1:5 significantly reduce the expression of transgenes after intravenous AAV administration. Particularly with systemic/intravenous administration of AAV, NAb completely blocks gene transduction at titers >1:10.

Currently, patients are excluded from clinical trials on the basis on their AAV neutralizing antibody titers, and it is anticipated that up to 30% of patients will not be eligible to receive approved AAV drugs based on their neutralizing antibody titers.

Various attempts to reduce the effect of pre-existing neutralizing antibodies to a selected AAV capsid on the ability to effectively treat patients with an rAAV having that capsid have been attempted. These approaches include the use of various immunosuppression regimens in conjunction with rAAV delivery.

The neonatal Fc receptor (FcRn) is a nonclassical major histocompatibility (MHC) class I molecule that consists of a unique transmembrane common β2-microglobulin (β2m). (Burmeister, W. P. Huber, A. H. & Bjorkman, P. J. Crystal structure of the complex of rat neonatal Fc receptor with Fc. Nature 372, 379-383 (1994), Burmeister, W. P. Gastinel, L. N. Simister, N. E., Blum, M. L. & Bjorkman, P. J. Crystal structure at 2.2 Å resolution of the MHC-related neonatal Fc receptor. Nature 372, 336-343 (1994); West, A. P. Jr. & Bjorkman, P. J. Crystal structure and immunoglobulin G (IgG) binding properties of the human major histocompatibility complex-related Fc receptor. Biochemistry 39, 9698-9708 (2000)). FcRn has been describes as playing a role in regulation of IgG and serum (SA) albumin levels in mammals. The three-dimensional structure of human FcRn has been described [V Oganesyan, et al, J Biol Chem., Vol 289, No. 11, pp 2812-78124 (March 2014). Inhibitors of FcRn have been suggested for roles in treating humorally-mediated autoimmune disorders. See, X Li and RP Kimberly, Expert Opin Ther Targets, 2014 March; 18(3): 335-350.

A need in the art exists for compositions and methods for gene therapy treatment of patients having neutralizing antibodies to AAV capsids.

### SUMMARY OF THE INVENTION

The compositions and regimens provided herein increase the population of patients that can be treated with a gene therapy vector by ablating the effect of neutralizing antibodies to a selected viral vector capsid, and thereby permitting effective delivery of an rAAV having the AAV capsid that carries the desired gene product.

A combination regimen for treating a patient with neutralizing antibodies to a viral vector, the regimen comprising administering a viral vector comprising an expression cassette comprising a nucleic acid sequence encoding a gene product for expression in a target cell and regulatory sequences which direct expression thereof in combination with a ligand which inhibits binding of human neonatal Fc receptor (FcRn) and immunoglobulin G (IgG). In certain embodiments, the viral vector is delivered systemically. In certain embodiments, the ligand is a peptide, protein, an RNAi sequence, or a small molecule. In certain embodiments, the protein is a monoclonal antibody, an immunoadhesin, a camelid antibody, a Fab fragment, an Fv fragment, or an scFv fragment. In certain embodiments, the recombinant viral vector is a recombinant adeno-associated virus, a recombinant adenovirus, a recombinant herpes simplex virus, or a recombinant lentivirus. In certain embodiments, the ligand is a monoclonal antibody which specifically inhibits FcRn-IgG binding without interfering with FcRn-albumin binding. In certain embodiments, the monoclonal antibody is nipocalimab (M281), rozanolixizumab (UCB7665); IMVT-1401, RVT-1401, HL161, HBM916, ARGX-113 (efgartigimod), SYNT001, SYNT002, ABY-039, or DX-2507, derivatives or combinations thereof. In certain embodiments, the ligand is delivered one to seven days prior to administration of the viral vector. In certain embodiments, the ligand is delivered daily. In certain embodiments, the ligand is dosed or administered on the same day the viral vector is administered. In certain embodiments, the ligand is dosed for one day to four weeks post-vector administration. In certain embodiments, the ligand is dosed via a different route of administration than the vector. In certain embodiments, the ligand is dosed orally. In certain embodiments, the viral vector is dosed intraperitoneally, intravenously, intramuscularly, intranasally, or intrathecally. In certain embodiments, the patient is predetermined to have a neutralizing antibody titer to the vector capsid which greater than 1:5 as determined in an in vitro assay. In certain embodiments, the patient has not previously received gene therapy prior to the delivery of the viral vector in combination with the inhibitory ligand such that the patient's pre-existing neutralizing antibodies are a result of wild-type infection. In certain embodiments, the patient has previously received gene therapy treatment prior to the delivery of the viral vector in combination with the inhibitory immunoglobulin construct. In certain embodiments, the regimen further comprises co-administering one or more of: (a) a steroid or combination of steroids and/or (b) an IgG-cleaving enzyme, (c) an inhibitor of Fc-IgE binding; (d) an inhibitor of Fc-IgM binding; (e) an inhibitor of Fc-IgA binding; and/or (f) gamma interferon.

In a further aspect, a method is provided for increasing the patient population for which gene therapy is effective. The method comprises co-administering to a patient from a population having a neutralizing antibody titer to a selected viral capsid or a serologically cross-reactive capsid which is greater than 1:5; (a) a recombinant virus having the selected viral capsid and a gene therapy expression cassette packaged therein; and (b) a ligand which specifically binds the neonatal Fc receptor (FcRn) prior to delivery of the gene therapy vector, wherein the ligand blocks the FcRN binding to immunoglobulin G (IgG) and permits effective amounts of the gene therapy product to be expressed in the patient.

In certain embodiments, a method for treating a patient with neutralizing antibodies to a capsid of a recombinant adeno-associated virus (rAAV) is provided. The method comprises administering the rAAV in combination with an anti-neonatal Fc receptor (FcRn) immunoglobulin construct, wherein said immunoglobulin construct specifically inhibits FcRn-immunoglobulin G (IgG) binding. In certain embodiments, the immunoglobulin construct is selected from a monoclonal antibody, an immunoadhesin, a camelid antibody, a Fab fragment, an Fv fragment, or an scFv fragment. In certain embodiments, the immunoglobulin construct specifically inhibits human FcRn-IgG binding without interfering with FcRn-albumin binding. In certain embodiments, the rAAV is delivered systemically, e.g., intravenously, intraperitoneally, intranasally, or via inhalation. In certain embodiments, the rAAV has a capsid selected from AAV1, AAV2, AAV3, AAV5, AAV7, AAV8, AAV9, AAVrh10, AAVhu37. In certain embodiments, the immunoglobulin construct comprises the CDRs of the heavy chain and/or the light chain of nipocalimab (M281). In certain embodiments, the immunoglobulin construct is a monoclonal antibody nipocalimab (M281). In certain embodiments, the immunoglobulin construct is delivered on the same day as the rAAV is administered. In certain embodiments, the immunoglobulin construct is delivered at least one day to four weeks post-rAAV administration. In certain embodiments, the immunoglobulin construct is delivered for four weeks to 6 months post-rAAV administration.

Other aspects and advantages of the invention will be readily apparent from the following detailed description of the invention.

### BRIEF DESCRIPTION OF THE DRAWINGS

FIGS. 1A and 1B show M281 mAb decreases hIgG and improves AAV-TT1 (Test Transgene 1) transduction in hFcRn mice treated with IVIG. FIG. 1A shows levels of serum human IgG at days 1 to 16 post IVIG treatment. FIG. 1B shows levels, represented in units (U), of transgene activity in serum post AAV8 vector delivery. Squares represent mice that received the M281 intravenous injection. Filled in squares represent mice in which IgG level reduction was observed. Empty squares represent mice in which the IgG level reduction was not observed.

FIGS. 2A and 2B show inhibition of FcRn by M281 reduced IVIG-derived NAb together with total hIgG and permitted for liver transduction following intravenous delivery of an AAV8 vector. FIG. 2A shows levels of serum human IgG (hIgG) at day 0 to day 5 post pre-treatment with IVIG. Administration of M281 and AAV vector are indicated by arrows. FIG. 2B shows TT1 levels in serum at day 0 to day 33 of study.

FIG. 3 shows a study design (Study 1 and Study 2) to evaluate the effects of blocking FcRn on NAb titer and AAV-TT2 (Test Transgene 2) transduction in non-human primates (NHPs).

FIGS. 4A to 4D show M281 infusion reduced pre-existing NAb titer together with IgG in NHPs (Study 1). FIG. 4A shows levels of serum rhesus macaque IgG (rhIgG), plotted as percent of day −5, where M281 administration is indicated by arrows on graph. FIG. 4B shows AAVhu68-non-neutralizing binding antibody (BAb) titer, wherein M281 administration is indicated by arrows on graph. FIG. 4C shows AAVhu68 neutralizing binding antibody (NAb) titer, wherein M281 administration is indicated by arrows on graph. FIG. 4D shows levels of serum albumin plotted as percent of day −5, wherein M281 administration is indicated by arrows on graph.

FIGS. 5A to 5B show M281 infusion reduced pre-existing NAb titer together with IgG in NHPs (Study 2). FIG. 5A shows levels of serum rhesus macaque IgG (rhIgG), plotted as percent of day −5, where administration of M281 (days −5, −4, and −3) and administration of AAV (day 0) are indicated by arrows on graph. FIG. 5B shows levels of serum albumin plotted as percent of day −5, wherein M281 administration is indicated by arrows on graph.

FIGS. 6A to 6B show AAV-binding antibody titer (Study 2). FIG. 6A shows AAVhu68-non-neutralizing binding antibody (BAb) titer, during study Day −15 to Day 0, wherein administration of M281 (days −5, −4, and −3) and administration of AAV (day 0). FIG. 6B shows AAVhu68-non-neutralizing binding antibody (BAb) titer, during study Day 0 to Day 30.

FIGS. 7A to 7E show vector genome biodistribution in various tissues harvested from Study 2, plotted as Genome Copy (GC) per micro-gram (μg) DNA. FIG. 7A shows vector genome biodistribution in heart. FIG. 7B shows vector genome biodistribution in skeletal muscle. FIG. 7C shows vector genome biodistribution in right lobe of liver. FIG. 7D shows vector genome biodistribution in left lobe of liver. FIG. 7E shows vector genome biodistribution in spleen.

FIGS. 8A and 8B shows results of in situ hybridization quantification examining TT2 mRNA expression levels in heart and liver tissues harvested from Study 2, plotted as positive area ratio. FIG. 8A shows results of in situ hybridization examining TT2 mRNA expression levels in liver tissue (left and right lobe) harvested from Study 2. FIG. 8B shows results of in situ hybridization examining TT2 mRNA expression levels in heart tissue (left, right ventricles and septum) harvested from Study 2.

FIG. 9 shows a study design to evaluate the effect of blocking pre-existing FcRn NAb titer following re-administration of AAV8.TT3 (test transgene 3) at a dose of 1×1013 GC/kg.

FIGS. 10A and 10B show results of AAV8.TT3 re-administration study, in which M281 administration reduced pre-existing NAb titer (AAV8) together with IgG in NHP (previously administered AAV8.TT3). FIG. 10A shows serum levels of rhesus macaque IgG (rhIgG), plotted as percent of day −5, where NHP was administered M281 at days −5, −4, −3, and −2 and AAV8.TT3 at day 0. FIG. 10B shows measured serum levels of M281 plotted as mg/mL.

FIGS. 11A and 11B shows results of another AAV8.TT3 study, in which M281 administration reduced pre-existing NAb titer (AAV8) together with IgG in NHP with pre-existing NAb+ (1:20) by natural infection. FIG. 11A shows total rhesus macaque IgG levels (rhIgG) plotted as percent of day −5, where NHP was administered M281 at days −5, −4, −3, and −2 and AAV8.TT3 at day 0. FIG. 11B shows serum M281 levels (hIgG) plotted as mg/mL and measured using ELISA.

FIG. 12 shows results of diminished TT1 activity levels in mice co-treated with IVIG at the time of AAV8.TT1 vector administration.

### DETAILED DESCRIPTION OF THE INVENTION

Regimens and compositions useful for the treatment of patients having neutralizing antibodies to the capsid of a viral vector selected for delivering a gene product to target cells/tissue in the patient are provided. In certain embodiments, the patient may be naïve to any therapeutic treatment with a selected viral vector and may have pre-existing immunity due to prior infections with a wild-type virus. In other embodiments, the patient may have neutralizing antibodies as a result of a prior treatment or vaccine. In certain embodiments, the patient may have neutralizing antibodies 1:1 to 1:20, or in excess of 1:2, in excess of 1:5, in excess of 1:10, in excess of 1:20, in excess of 1:50, in excess of 1:100, in excess of 1:200, in excess of 1:300 or higher. In certain embodiments, a patient has neutralizing antibodies in the range of 1:1 to 1:200, or 1:5 to 1:100, or 1:2 to 1:20, or 1:5 to 1:50, or 1:5 to 1:20. In certain embodiments, a patient receives a single anti-FcRn ligand (e.g., anti-FcRn antibody) as the sole agent to modulate FcRn-IgG binding and to permit effective vector delivery. In other embodiments, a patient may receive a combination of one or more anti-FcRn ligands and a second component (e.g., an Fc receptor down-regulator (e.g., interferon gamma), an IgG enzyme, or another suitable component). Such combinations may be particularly desirable for patients having particularly high neutralizing antibody levels (e.g., in excess of 1:200). In certain embodiments, the compositions comprising anti-FcRn ligands and the regimens and co-administration are utilized during systemic delivery of viral vectors. However, the invention is not so limited, as described in more detail herein.

As used herein, the term “FcRn” refers a neonatal Fc receptor that binds to the Fc region of an immunoglobulin (IgG) antibody. An exemplary FcRn is human FcRn having UniProt ID No. P55899 (SEQ ID NO: 45). Human FcRn is believed to be responsible for maintaining the half-life of IgG by binding and trafficking constitutively internalized IgG back to the cell surface for the recycling of IgG. Unless otherwise specified, “FcRn” refers to a patient's native FcRn.

As used herein, the term “immunoglobulin G” or “IgG” refers to any member of a class of antibodies composed of four different subtypes or subclasses of IgG molecules, designated IgG1, IgG2, IgG3 and IgG4. Unless otherwise specified an anti-FcRn ligand selected for use in the compositions, regimens and combinations provided herein may bind any subtype of a patient's IgG neutralizing antibody (NAb). In certain embodiments, an anti-Fc ligand may be selected which binds a subset of the NAb IgG subclasses, e.g., only IgG1, or IgG1, IgG2, or IgG3 or IgG4, only IgG4, or other combinations. While it is currently believed that pre-existing neutralizing antibodies to selected outer capsids (for non-enveloped vectors) or envelope proteins of a viral vectors (e.g., a capsid protein of an AAV) are primarily of the IgG class (or subclasses thereof), in certain embodiments, the compositions and combinations provided herein containing inhibitory ligands are useful for inhibiting neutralizing antibodies of other immunoglobulin classes, e.g., pre-existing neutralizing antibodies against an AAV capsid or a serologically cross-reactive capsid

In certain embodiments, the compositions provided herein are particularly well suited for use when the viral vector (e.g., gene therapy vector) is being delivered systemically. As used herein, “systemic delivery” encompasses any suitable route of delivery, including, without limitation, intraperitoneal, intramuscular, subcutaneous, intravenous, oral, direct administration to an organ (e.g., heart, liver), excluding the eye (e.g., intravitreal, subretinal), brain and other parts of the central nervous system (e.g., intrathecal). However, there are certain embodiments where delivery of an FcRn blocking ligand composition as provided herein is desired for use in conjunction with non-systemic vector-based gene therapy, e.g., vector administered directly to the eye (e.g., subretinal, intravitreal), brain, or another part of the CNS.

As used herein, the term “vector” refers to any molecule or moiety which transports, transduces or otherwise acts as a carrier of a heterologous molecule such as the polynucleotides. A “viral vector” is a vector which comprises one or more polynucleotide regions encoding or comprising a molecule of interest, e.g., a transgene, a polynucleotide encoding a polypeptide or multi-polypeptide or a modulatory nucleic acid. The viral vectors may be produced recombinantly. The examples herein demonstrate the methods for co-administering an anti-FcRN ligand with a recombinant adeno-associated virus (AAV) capsid to which the selected patient has neutralizing antibodies.

However, the methods and compositions may be utilized in patients having neutralizing antibodies to an adenovirus capsid protein (for treatment with a recombinant adenovirus), a herpes simplex virus (for treatment with a recombinant herpes simplex virus vector), or a lentivirus (for treatment with a recombinant lentivirus). Within each of these vector categories, neutralizing antibodies may be serologically specific, but within this specificity may be viruses having the same capsid source or different capsid source which is serologically cross-reactive with the capsid. Different virus capsids within each of the virus types: AAV, adenovirus, HSV, or lentivirus, may be serologically distinct or serologically cross-reactive. For example, patient to be administered an rAAV8 carrying a desired transgene will be screened for neutralizing antibodies to AAV8.

As used herein, a “neutralizing antibody” or “NAb” binds specifically to a viral capsid or envelope and interferes with the infectivity of the virus or a recombinant viral vector having the viral capsid or envelope, thus preventing the recombinant viral vector from delivering effective amounts of a gene product encoded by an expression cassette in its vector genome. Various methods for assessing neutralizing antibodies in a patient's sera may be utilized. The term method and assay may be used interchangeably. As used herein, the term “neutralization assay” and “serum virus neutralization assay” refers to a serological test to detect the presence of systemic antibodies that may prevent infectivity of a virus. Such assays may also qualitatively or quantitatively discern the binding capacity (e.g., magnitude) or efficiency of the antibodies to neutralize a target Immunological assays may include enzyme immunoassay (EIA), radioimmunoassay (RIA), which uses radioactive isotopes, fluoroimmunoassay (FIA) which uses fluorescent materials, chemiluminescent immunoassay (CLIA) which uses chemiluminescent materials and counting immunoassay (CIA) which employs particle-counting techniques, other modified assays such as western blot, immunohistochemistry (IHC) and agglutination. One of the most common enzyme immunoassays is enzyme-linked immunosorbent assay (ELISA).

Example of suitable methods include those described, e.g., R Calcedo, et al, Journal Infectious Diseases, 2009, 199:381-290; GUO, et al., “Rapid AAV Neutralizing Antibody Determination with a Cell-Binding Assay”, Molecular Therapy: Methods & Clinical Development Vol. 13 Jun. 2019, T. Ito et al, “A convenient enzyme-linked immunosorbent assay for rapid screening of anti-adeno-associated virus neutralizing antibodies”, Ann Clin Biochem 2009; 46: 508-510; US 201810356394A2 (Voyager Therapeutics). Additionally, commercial kits exist (see, e.g., Athena Diagnostics, Invitrogen, ThermoFisher.com; Covance).

The neutralization ability of an antibody is usually measured via the expression of a reporter gene such as luciferase or GFP. In order to determine and compare the activity of a neutralizing antibody, the antibody tested should display a neutralizing activity of 50% or more in one of the neutralization assays described herein. In some examples, neutralizing capacity is determined by measuring the activity of a reporter gene product (e.g., luciferase, GFP). The neutralizing capacity of an antibody to a specific viral vector may be at least 50%, e.g., at least 55%, 60%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99%.

Currently, many clinical trials require testing a potential patient for the existing of NAbs to the capsid (or envelope) of the viral vector being tested. Certain clinical trials currently use a NAb titer of 1:20 or greater, 1:10 or greater, or 1:5 or greater as an exclusion condition for treatment in the trial. This is particularly important where the viral vector will be delivered systemically. However, the compositions and methods provided herein may permit patients which fall within one, two or all of these exclusion criteria to receive effective gene therapy (or vaccine) treatment. Effective gene transfer may be determined using the standards selected for the patient population not having the preselected, NAb titer. For example, effective gene transfer may be determined by measuring transgene expression, disease biomarkers, reduction of symptoms of the disease, reduction of disease progression, and/or other preselected determinants of improved clinical sequalae.

As used herein, the term “NAb titer” a measurement of how much neutralizing antibody (e.g., anti-AAV Nab) is produced which neutralizes the physiologic effect of its targeted epitope (e.g., an AAV). Anti-AAV NAb titers may be measured as described in, e.g., Calcedo, R., et al., Worldwide Epidemiology of Neutralizing Antibodies to Adeno-Associated Viruses. Journal of Infectious Diseases, 2009. 199(3): p. 381-390, which is incorporated by reference herein.

The method comprises administering to a subject a suspension of a vector as described herein. In one embodiment, the method comprises administering to a subject a suspension of a rAAV as described herein in a formulation buffer.

The composition(s) and method(s) permit treatment of a subject (human patient) in need thereof with a vector. In particularly desirable embodiments,

### 1. FcRn Ligand

As used herein, an “FcRn ligand” is any moiety (e.g., without limitation, peptide, protein, antibody, a shRNA, RNAi, a nucleic acid encoding a peptide, protein, or antibody, or small molecule drug) which blocks or significantly reduces binding between human neonatal Fc receptor (FcRn) and a patient's neutralizing antibodies. In desired embodiments, the ligand may be referred to herein as “anti-FcRn”. In certain embodiments, the FcRn ligand blocks FcRn binding to a patient's NAbs without blocking FcRn binding to albumin. This may be referred to herein as an FcRn-IgG blocking ligand, an FcRn-NAb blocking ligand, or an anti-FcRn ligand.

As used herein, the term “inhibit IgG binding to FcRn” refers to the ability of a ligand to block or inhibit the binding of IgG (e.g., IgG1) to a patient's native FcRn (e.g., human FcRn in a human patient). In some embodiments, the ligand binds FcRn, for example, at the site on human FcRn to which IgG binds. Thus, the ligand inhibits the binding of a patient's IgG autoantibodies to FcRn. In some embodiments, the ligand substantially or completely inhibits binding to IgG. In some embodiments, the binding of IgG is reduced by 10%, 20%, 30%, 50%, 70%, 80%, 90%, 95%, or even 100%.

As used herein specifically inhibits FcRn without blocking or interfering with albumin levels refers to the characteristic of the selected anti-FcRn ligand and its ability to specifically reduce binding of anti-AAV neutralizing antibodies to the FcR. By “specifically” is meant that post-treatment with the anti-FcRn antibody regimen provided herein, the patient retains at least the minimum levels of serum albumin necessary.

Preferably, patient albumin levels remain within a normal range, e.g., about 3.4 g/dL to about 5.5 g/dL (34 to 54 g/L), but may be characterized by mild depletion (e.g., 2.8 to 3.4 g/dL) to moderate albumin depletion (2 g/dL to 2.7 g/dL). Patients having mild, moderate or severe albumin depletion (e.g., less than 3 g/dl), may still be candidates for the therapeutic regimen. In certain embodiments, albumin replacement therapy (e.g., delivered in a intravenous infusion) may be further co-administered with the regimen provided herein.

Anti-FcRn Immunoglobulins

In certain embodiments, a monoclonal antibody is selected which has the complementarity determining regions (CDRs) of heavy (H) chain CDR H1 [SEQ ID NO: 16 or a sequence at least 99% identical thereto, CDR H2, [SEQ ID NO:18] or a sequence at least 99% identical thereto, CDR H3 [SEQ ID NO: 20] or a sequence at least 99% identical thereto is selected. In certain embodiments, the full-length heavy chain of N027 of WO 2016/124521 is provided. See, e.g., SEQ ID NO: 8, or a sequence at least 95% identical thereto. In certain embodiments, there is no more than 1 amino acid change in any of CDR H1, H2 and/or H3. In certain embodiments, there is no more than 1 to 4 amino acid changes in the heavy chain. In certain embodiments, the CDRs of the heavy chain are selected for use, but are engineered into a different antibody scaffold and different heavy chain constant regions are selected. In certain embodiments, a monoclonal antibody is selected which has the light chain CDRs of the CDR L1 [SEQ ID NO: 10 or a sequence at least 99% identical thereto, CDR L2, [SEQ ID NO:12] or a sequence at least 99% identical thereto, CDR L3 [SEQ ID NO: 14] or a sequence at least 99% identical thereto is selected. In certain embodiments, the full-length light chain of N027 of WO 2016/124521 is provided. See, e.g., SEQ ID NO: 7, or a sequence at least 95% identical thereto. In certain embodiments, there is no more than 1 amino acid change in any of CDR L1, L2 and/or L3. In certain embodiments, there is no more than 1 to 4 amino acid changes in the light chain. In certain embodiments, the CDRs of the light chain are selected for use, but are engineered into a different antibody scaffold and different light chain constant regions are selected. In certain embodiment, the monoclonal antibody has the heavy chains of SEQ ID NO: 4 or 8 and the light chains of SEQ ID NO: 2 or 7, or a sequence at least 95% identical thereto, at least 97% identical thereto, or at least 99% identical thereto. In certain embodiments, the CDRs of the light chain are further selected from CDR L3 variant of SEQ ID NO: 41 and/or CDR L2 variant of SEQ ID NO: 42, or a sequence at least 99% identical thereto. In certain embodiments, the CDRs of the heavy chain are further selected from CDR H1 variants of SEQ ID NOs: 21, 22, and 43, or a sequence at least 99% identical thereto, CDR H2 variants of SEQ ID NO: 23, 24, 25, and 44, or a sequence at least 99% identical thereto.

As used herein, the terms “complementary determining regions” and “CDRs” refer to the regions of an antibody variable domain which are hypervariable in sequence and/or form structurally defined loops. A CDR is also known as a hypervariable region. The light chain and heavy chain variable regions each has three CDRs. The light chain variable region contains CDR L1, CDR L2, and CDR L3. The heavy chain variable region contains CDR H1, CDR H2, and CDR H3. Each CDR may include amino acid residues from a complementarity determining region as defined by Kabat (i.e., about residues 24 to about 34 (CDR L1), about 50 to about 56 (CDR L2) and about 89 to about 97 (CDR L3) in the light chain variable region and about residues 31 to about 35 (CDR H1), about 50 to about 65 (CDR H2) and about 95 to about 102 (CDR H3) in the heavy chain variable region.

As used herein, the terms “variable region” and “variable domain” refer to the portions of the light and heavy chains of an antibody that include amino acid sequences of complementary determining regions (CDRs, e.g., CDR L 1, CDR L2, CDR L3, CDR H 1, CDR H2, and CDR H3) and framework regions (FRs). The amino acid positions assigned to CDRs and FRs are defined according to Kabat (Sequences of Proteins of Immunological Interest, 5th Ed. Public Health Service, National Institutes of Health, Bethesda, Md. (1991)). Using this numbering system, the actual linear amino acid sequence may contain fewer or additional amino acids corresponding to a shortening of, or insertion into, a CDR (defined further herein) or FR (defined further herein) of the variable region. For example, a heavy chain variable region may include a single inserted residue (i.e., residue 52a according to Kabat) after residue 52 of CDR H2 and inserted residues (i.e., residues 82a, 82b, 82c, etc. according to Kabat) after residue 82 of heavy chain FR. The Kabat numbering of residues may be determined for a given antibody by alignment at regions of homology of the sequence of the antibody with a “standard” Kabat numbered sequence.

The term “immunoglobulin” is used herein to include antibodies, functional fragments thereof, and immunoadhesins. In certain embodiments, these are also termed herein “immunoglobulin constructs” or “antibody constructs”. Antibodies may exist in a variety of forms including, for example, polyclonal antibodies, monoclonal antibodies, camelized single domain antibodies, intracellular antibodies (“intrabodies”), recombinant antibodies, multispecific antibody, antibody fragments, such as, Fv, Fab, F(ab)₂, F(ab)₃, Fab′, Fab′-SH, F(ab′)2, single chain variable fragment antibodies (scFv), tandem/bis-scFv, Fc, pFc′, scFvFc (or scFv-Fc), disulfide Fv (dsfv), bispecific antibodies (bc-scFv) such as BiTE antibodies; camelid antibodies, resurfaced antibodies, humanized antibodies, fully human antibodies, single-domain antibody (sdAb, also known as NANOBODY®), multi-domain antibodies (mdAb), chimeric antibodies, chimeric antibodies comprising at least one human constant region, and the like.

The anti-FcRn immunoglobulin constructs described herein may be produced in any suitable production system in vitro, purified, and formulated in a suitable composition delivery to the patient as described herein. Optionally, these constructs may contain one or more immunoglobulin constructs. Optionally, these constructs (e.g., monoclonal antibodies) may be formulated separately from a viral vector. In other embodiments, the constructs may be formulated and delivered with a viral vector.

In other embodiments, another monoclonal antibody may be selected, or used in combination. Examples of such antibodies may include, e.g., rozanolixizumab (UCB7665) (UCB SA); IMVT-1401, RVT-1401 (HL161), HBM9161 (all form HanAll BioPhrma Co. Ltd), nipocalimab (M281) (Momenta Pharmaceuticals Inc), ARGX-113 (efgartigimod) (Argenx S.E.), orilanolimab (ALXN 1830, SYNT001, Alexion Pharmaceuticals Inc), SYNT002, ABY-039 (Affibody AB), or DX-2507 (Takeda Pharmaceutical Co. Ltd), combinations thereof, or one of these antibodies in combination with another ligand. Alternatively, other antibody constructs may be derived from these antibodies, among others.

Pharmaceutical compositions of the invention that contain one or more anti-FcRn antibody constructs as therapeutic proteins may be formulated for intravenous administration, parenteral administration, subcutaneous administration, intramuscular administration, intra-arterial administration, intrathecal administration, or intraperitoneal administration. In particular, intravenous administration is preferred. The pharmaceutical composition may also be formulated for, or administered via, oral, nasal, spray, aerosol, rectal, or vaginal administration. For injectable formulations, various effective pharmaceutical carriers are known in the art. The dosage of the pharmaceutical compositions of the invention depends on factors including the route of administration and physical characteristics, e.g., age, weight, general health, of the subject. A pharmaceutical composition may include a dosage of an anti-FcRn antibody construct of ranging from 0.01 to 500 mg/kg (e.g., 0.01, 0.1, 0.2, 0.3, 0.4, 0.5, 1, 2, 3, 4, 5, 10, 15, 20, 25, 30, 35, 40, 45, 50, 100, 150, 200, 250, 300, 350, 400, 450, or 500 mg/kg) and, in a more specific embodiment, about 1 to about 100 mg/kg and, in a more specific embodiment, about 1 to about 50 mg/kg, in a more specific embodiment, about 15 to about 30 mg/kg. The dosage may be adapted by the physician in accordance with conventional factors such as the extent of the disease and different parameters of the subject. The pharmaceutical compositions are administered in a manner compatible with the dosage formulation. The pharmaceutical compositions are administered in a variety of dosage forms, e.g., intravenous dosage forms, subcutaneous dosage forms, and oral dosage forms (e.g., ingestible solutions, drug release capsules). Generally, therapeutic proteins are dosed at 1 to 100 mg/kg, e.g., 1 to 50 mg/kg. In certain embodiments, these compositions may be dosed in an ascending or a descending dose for a pre-determined number of days (e.g., 3 to 7 days) or over another pre-selected period of time. Optionally, a ligand (e.g., an anti-FcRn antibody) may be engineered into a suitable delivery vector (e.g., an rAAV or another viral vector) and delivered in a suitable amount to express protein levels in the amounts above.

Pharmaceutical compositions that contain an anti-FcRn antibody construct may be administered to a subject in need thereof, for example, one or more times (e.g., 1-10 times or more) daily, weekly, monthly, biannually, annually, or as medically necessary. Dosages may be provided in either a single or multiple dosage regimens.

Anti-FcRn Peptide and Proteins

Optionally, additionally or alternatively to an FcRn antibody construct described above, a suitable anti-FcRn ligand may be a peptide or protein construct binding human FcRn so as to inhibit IgG binding. Examples may include, e.g., SYN1436 (Biogen), a 26-amino acid peptide which binds to FcRn to block its interaction with IgG, which is a dimer of SYN 1327 (SEQ ID NO: 30), or a modified variant thereof (SEQ ID NO: 31), or a non-truncated and non-dimerized peptide variant thereof. SYN746 (SEQ ID NO: 29). See, also, U.S. Pat. No. 9,527,890. Furthermore, an example may include an FcRN affinity binding molecule, e.g., Z_{FcRn} having 58 amino acids and a folded anti-parallel three-helix bundle structure, or an Z_{FcRn}, fusion protein, such as a Z_{FcRn}-albumin binding domain (ABD) fusion protein. Wherein, Z_{FcRn} peptides may comprise ZFcRn-2 with amino acid sequence of SEQ ID NO: 26, ZFcRn-4 with amino acid sequence of SEQ ID NO: 27, and/or ZFcRn-16 with amino acid sequence of SEQ ID NO: 28 (Seijsing, J., et al., 2014, PNAS, 111(48):1710-17115). Other suitable anti-FcRn ligands may include, e.g., QRFCTGHFGGLYPCNGP (SEQ ID NO: 32), GGGCVTGHFGGIYCNYQ (SEQ ID NO: 33), KIICSPGHFGGMYCQGK (SEQ ID NO: 34), PSYCIEGHIDGIYCFNA (SEQ ID NO: 35), and/or NSFCRGRPGHFGGCYLF (SEQ ID NO: 36). See, e.g., WO 2007/098420 A2. Furthermore, an example of a suitable protein construct may include DX-2504 light chain (SEQ ID NO: 37), DX-2504 heavy chain (SEQ ID NO: 38), DX-2507 light chain (SEQ ID NO: 39) and/or DX-2507 heavy chain (SEQ ID NO: 40). See, also U.S. Pat. No. 9,359,438 B2.

A pharmaceutical composition may include a dosage of an anti-FcRn peptide or protein ranging from 0.01 to 500 mg/kg (e.g., 0.01, 0.1, 0.2, 0.3, 0.4, 0.5, 1, 2, 3, 4, 5, 10, 15, 20, 25, 30, 35, 40, 45, 50, 100, 150, 200, 250, 300, 350, 400, 450, or 500 mg/kg) and, in a more specific embodiment, about 1 to about 100 mg/kg and, in a more specific embodiment, about 1 to about 50 mg/kg.

Protein and Peptide Production

A nucleic acid sequence encoding the amino acid sequence of an anti-FcRn protein or peptide (e.g., an immunoglobulin, immunoglobulin construct, antibody, antibody construct) may be prepared by a variety of methods known in the art. The sequence listing provides the nucleic acid sequence used to express the light chain and the heavy chain of the antibody expressed in vitro and used in the examples. These sequences include, e.g., SEQ ID NO: 5 or a sequence at least 90% identical thereto encoding M281 light chain having the amino acid sequence of SEQ ID NO: 7, SEQ ID NO: 6 or a sequence at least 90% identical thereto encoding M281 heavy chain amino acid sequence of SEQ ID NO: 8, SEQ ID NO: 9 or a sequence at least 90% identical thereto encoding M281 CDR-L1 having the amino acid sequence of SEQ ID NO: 10, SEQ ID NO: 11 or a sequence 90% identical thereto encoding M281 CDR-L2 having the amino acid sequence of SEQ ID NO: 12, SEQ ID NO: 13 or a sequence 90% identical thereto encoding M281 CDR-L3 having the amino acid sequence of SEQ ID NO: 14, SEQ ID NO: 15 or a sequence at least 90% identical thereto encoding M281 CDR-H1 having the amino acid sequence of SEQ ID NO: 16, SEQ ID NO: 17 or a sequence 90% identical thereto encoding M281 CDR-H2 having the amino acid sequence of SEQ ID NO: 18, SEQ ID NO: 19 or a sequence 90% identical thereto encoding M281 CDR-H3 having the amino acid sequence of SEQ ID NO: 20. Other nucleic acid sequences may include those encoding one or more of M281 CDR-H1 variants having an amino acid sequence of SEQ ID NO: 21 or SEQ ID NO: 22, M281 CDR-H2 variants having an amino acid sequence of SEQ ID NO: 23, or SEQ ID NO: 24, or SEQ ID NO: 25.

Other nucleic acid sequences may include those encoding one or more of the proteins and peptides having the amino acid sequence of SEQ ID NO: 26 (Z FcRn2), SEQ ID NO: 27 (Z FcRn-4); SEQ ID NO: 28 (Z FcRn-16), SYN746 (SEQ ID: 29), SEQ ID NO: 30 (SYN 1327), modified SYN1327 (SEQ ID NO: 31), 98420-p1 (SEQ ID NO: 32), 98420-p2 (SEQ ID NO: 33), 98420-p3 (SEQ ID NO: 34), 98420-p4 (SEQ ID NO: 35), 98420-p5 (SEQ ID NO: 36), DX-2504-LC (SEQ ID NO: 37), DX-2504-HC (SEQ ID NO: 38), DX-2507-LC (SEQ ID NO: 39), DX-2507-HC (SEQ ID NO: 40), DX-CDR-L3 (SEQ ID NO: 41), DX-CDR-L2 (SEQ ID NO: 42), DX-CDR-H1 (SEQ ID NO: 43), or DX-CDR-H2 (SEQ ID NO: 44).

A nucleic acid molecule encoding an anti-FcRn ligand may be obtained using standard techniques, e.g., gene synthesis. Alternatively, a nucleic acid molecule encoding a wild-type anti-FcRn ligand (e.g., antibody) may be mutated to contain specific amino acid substitutions using standard techniques in the art, e.g., QuikChange™ mutagenesis. Nucleic acid molecules can be synthesized using a nucleotide synthesizer or PCR techniques. The nucleic acid sequences encoding anti-FcRn antibodies or other ligands may be inserted into a vector capable of replicating and expressing the nucleic acid molecules in prokaryotic or eukaryotic host cells. Many vectors suitable for in vitro protein or peptide production are available in the art and can be used. Each vector may contain various components that may be adjusted and optimized for compatibility with the particular host cell. For example, the vector components may include, but are not limited to, an origin of replication, a selection marker gene, a promoter, a ribosome binding site, a signal sequence, the nucleic acid sequence encoding protein of interest, and a transcription termination sequence. In other embodiments, a vector may be designed for in vivo production of a ligand such as anti-FcRn antibody. Such vectors may be selected from any suitable vector, e.g., an rAAV or other viral vector such as those described in connection with an rAAV encoding a gene product.

In some embodiments, the vector used for production of an anti-FcRn antibody is a plasmid which comprises a nucleic acid sequence of SEQ ID NO:1, encoding for the amino acid sequence of an anti-FcRn protein M281-LC of SEQ ID NO: 2. In some embodiments, plasmid comprising a vector used for production of anti-FcRn protein or peptide comprises a nucleic acid sequence of SEQ ID NO:3 or a sequence at least 90% identical thereto which encodes the amino acid sequence of an anti-FcRn protein M281-HC of SEQ ID NO: 4.

In some embodiments, mammalian cells are used as host cells. Examples of mammalian cell types include, but are not limited to, human embryonic kidney (HEK) (e.g., HEK293, HEK 293F), Chinese hamster ovary (CHO), HeLa, COS, PC3, Vero, MC3T3, NSO, Sp2/0, VERY, BHK, MDCK, W 138, BT483, Hs578T, HTB2, BT20, T47D, NSO (a murine myeloma cell line that does not endogenously produce any immunoglobulin chains), CRL7030, and HsS78Bst cells. In other embodiments, E. coli cells are used as host cells for the invention. Different host cells have characteristic and specific mechanisms for the posttranslational processing and modification of protein products. Appropriate cell lines or host systems may be chosen to ensure the correct modification and processing of the anti-FcRn antibody (or other ligand) expressed. The above-described expression vectors may be introduced into appropriate host cells using conventional techniques in the art, e.g., transformation, transfection, electroporation, calcium phosphate precipitation, and direct microinjection.

Once the vectors are introduced into host cells for protein production, host cells are cultured in conventional nutrient media modified as appropriate for inducing promoters, selecting transformants, or amplifying the genes encoding the desired sequences. Methods for expression of therapeutic proteins are known in the art, see, for example, Paulina Balbas, Argelia Lorence (eds.) Recombinant Gene Expression: Reviews and Protocols (Methods in Molecular Biology), Humana Press; 2nd ed. 2004 (Jul. 20, 2004) and Vladimir Voynov and Justin A. Caravella (eds.) Therapeutic Proteins: Methods and Protocols (Methods in Molecular Biology) Humana Press; 2nd ed. 2012 (Jun. 28, 2012).

Host cells used to produce the anti-FcRn ligands (e.g., antibodies or other peptides or proteins) may be grown in media known in the art and suitable for culturing of the selected host cells. Suitable media for mammalian host cells may include, e.g., Minimal Essential Medium (MEM), Dulbecco's Modified Eagle's Medium (DMEM), Expi293™ Expression Medium, DMEM with supplemented fetal bovine serum (FBS), and RPMI-1640. Examples of suitable media for bacterial host cells include Luria broth (LB) plus necessary supplements, such as a selection agent, e.g., ampicillin. Host cells are cultured at suitable temperatures, such as from about 20° C. to about 39° C., e.g., from 25° C. to about 37° C., preferably 37° C., and CO₂ levels. The pH of the medium is generally from about 6.8 to 7.4, e.g., 7, depending mainly on the host organism. If an inducible promoter is used in the expression vector of the invention, protein expression is induced under conditions suitable for the activation of the promoter. Protein recovery typically involves disrupting the host cell, generally by such means as osmotic shock, sonication, or lysis. Once the cells are disrupted, cell debris may be removed by centrifugation or filtration. The proteins may be further purified. An anti-FcRn antibody may be purified by any method known in the art of protein purification, for example, by protein A affinity, other chromatography (e.g., ion exchange, affinity, and size-exclusion column chromatography), centrifugation, differential solubility, or by any other standard technique for the purification of proteins (see Process Scale Purification of Antibodies, Uwe Gottschalk (ed.) John Wiley & Sons, Inc., 2009). In some instances, an anti-FcRn antibody (or other ligand) can be conjugated to marker sequences, such as a peptide to facilitate purification. An example of a marker amino acid sequence is a hexa-histidine peptide (His-tag), which binds to nickel-functionalized agarose affinity column with micromolar affinity. Other peptide tags useful for purification include, but are not limited to, the hemagglutinin “HA” tag, which corresponds to an epitope derived from the influenza hemagglutinin protein.

In Vivo Expression of Anti-FcRn Proteins or Peptides

In certain embodiments, nucleic acid sequences encoding anti-FcRn immunoglobulins, peptides and/or proteins are delivered to a subject (e.g., a human patient), allowing the subject to produce the anti-FcRn immunoglobulins, peptides and/or proteins (e.g., anti-FcRn antibodies). In the context of therapy, this may be accomplished by administrating a viral or non-viral vector carrying these nucleic acid sequences. Such a vector may be a replication-incompetent adeno-associated virus (AAV) or another viral vector, e.g., a retroviral vector, adenoviral vector, poxviral vector (e.g., vaccinia viral vector, such as Modified Vaccinia Ankara (MVA)), or alphaviral vector) containing a nucleic acid molecule encoding the anti-FcRn ligand under the control of regulatory sequences which direct expression thereof in the host cell. Optionally, the regulatory sequences may include a regulatable promoter which permits control of expression of the anti-FcRn ligand through dosing with pharmacologic moiety (e.g., a rapalog or rapamycin). In other embodiments, the regulatory sequences may another suitable promoter, e.g., a constitutive promoter, a tissue-specific promoter, or another desired type of promoter. In certain embodiments, the anti-FcRn ligand is delivered via the same vector as delivers the coding sequence for the therapeutic or vaccine gene product(s). The vector, once inside a cell of the subject (e.g., by transformation, transfection, electroporation, calcium phosphate precipitation, direct microinjection, infection, etc) will promote expression of the anti-FcRn ligand (e.g., antibody construct), which is then secreted from the cell.

In certain embodiments, the nucleic acid molecule encoding the anti-FcRn ligand (e.g., anti-FcRn antibody) under the control of regulatory sequences which direct expression thereof in the host cell is a nucleic acid sequence in an expression cassette.

In certain embodiments, receptor-targeted nanoparticles may be used, wherein the nanoparticle comprises encapsulated nucleic acid sequence encoding the anti-FcRn ligand (e.g., anti-FcRn antibody) under the control of regulatory sequences which direct expression thereof in the host cell. For example, receptor-targeted nanoparticles may be used deliver mRNA or other active agents including peptides. Examples of such nanoparticles are provided, e.g., in US2018/0021455A1.

Small Molecule Inhibitors

In certain embodiments, a small molecule inhibitor of FcRn-IgG binding may be selected. See, e.g., Z Wang, et al, “Discovery and structure-activity relationships of small molecules that block the human immunoglobulin G-human neonatal Fc receptor (hIgG-hFcRn) protein-protein interaction”, Bioorganic & Medicinal Chemistry Letters, Vol 23, Issue 5, 1 Mar. 2013, pp. 1253-1256.

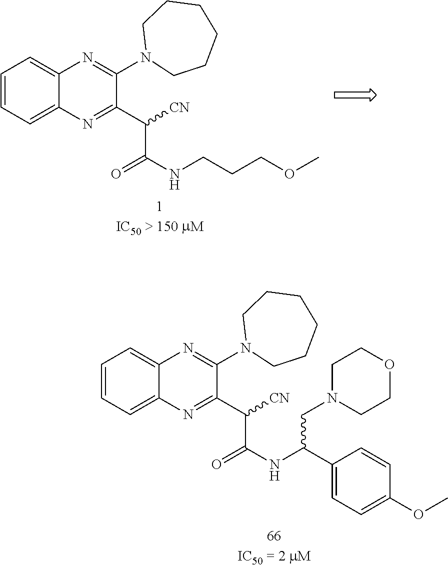 [SMILES: COCCCNC(=O)C(C#N)c1nc2ccccc2nc1N1CCCCCC1 ; COc1ccc(C(CN2CCOCC2)NC(=O)C(C#N)c2nc3ccccc3nc2N2CCCCCC2)cc1]

Optionally, an antibody or other ligand to another Fc receptor may be used in combination with an FcRn ligand as provided herein. Such other receptors may include, e.g., a receptor for IgA (e.g., Fcα (CD89), a receptor for IgE (FcεRI), a receptor for IgM (FCμR). See, e.g., X Li and R P Kimberly, Expert Opin Ther Targets, 2014 March; 18(3): 335-350, which is incorporated herein by reference.

### 2. Expression Cassette

The regimen and methods for treating a patient with neutralizing antibodies to a viral vector involve administering a viral vector and the anti-FcRn-IgG ligand. The viral vectors comprise an expression cassette comprising a nucleic acid sequence encoding a gene product for expression in a target cell and regulatory sequences which direct expression thereof in the target cell when administered to a patient without neutralizing antibodies to the viral vector or when administered with the method provided herein.

As used herein, an “expression cassette” refers to a nucleic acid molecule which comprises a biologically useful nucleic acid sequence (e.g., a gene cDNA encoding a protein, enzyme or other useful gene product, mRNA, etc.) and regulatory sequences operably linked thereto which direct or modulate transcription, translation, and/or expression of the nucleic acid sequence and its gene product. As used herein, “operably linked” sequences include both regulatory sequences that are contiguous or non-contiguous with the nucleic acid sequence and regulatory sequences that act in trans or cis nucleic acid sequence. Such regulatory sequences typically include, e.g., one or more of a promoter, an enhancer, an intron, a Kozak sequence, a polyadenylation sequence, and a TATA signal. The expression cassette may contain regulatory sequences upstream (5′ to) of the gene sequence, e.g., one or more of a promoter, an enhancer, an intron, etc., and one or more of an enhancer, or regulatory sequences downstream (3′ to) a gene sequence, e.g., 3′ untranslated region (3′ UTR) comprising a polyadenylation site, among other elements. In certain embodiments, the regulatory sequences are operably linked to the nucleic acid sequence of a gene product, wherein the regulatory sequences are separated from nucleic acid sequence of a gene product by an intervening nucleic acid sequences, i.e., 5′-untranslated regions (5′UTR). In certain embodiments, the expression cassette comprises nucleic acid sequence of one or more of gene products. In some embodiments, the expression cassette can be a monocistronic or a bicistronic expression cassette. In other embodiments, the term “transgene” refers to one or more DNA sequences from an exogenous source which are inserted into a target cell.

Typically, such an expression cassette can be used for generating vector genome for a viral vector and contains the coding sequence for the gene product described herein flanked by packaging signals of the viral genome and other expression control sequences such as those described herein. In certain embodiments, a vector genome may contain two or more expression cassettes. Optionally, an expression cassette (and a vector genome) may comprise one or more dorsal root ganglion (drg)-miRNA targeting sequences in the UTR, e.g., to reduce drg-toxicity and/or axonopathy. See, e.g., PCT/US2019/67872, filed Dec. 20, 2019 and now published as WO 2020/132455, US Provisional Patent Application No. 63/023,593, filed May 12, 2020, and U.S. Provisional Patent Application No. 63/038,488, filed Jun. 12, 2020, all entitled “Compositions for Drg-Specific Reduction of Transgene Expression”, which are incorporated herein in their entireties.

As used herein, a “vector genome” refers to the nucleic acid sequence packaged inside a parvovirus (e.g., rAAV) capsid which forms a viral particle. Such a nucleic acid sequence contains AAV inverted terminal repeat sequences (ITRs). In the examples herein, a vector genome contains, at a minimum, from 5′ to 3′, an AAV 5′ ITR, coding sequence(s) (i.e., transgene(s)), and an AAV 3′ ITR. ITRs from AAV2, a different source AAV than the capsid, or other than full-length ITRs may be selected. In certain embodiments, the ITRs are from the same AAV source as the AAV which provides the rep function during production or a transcomplementing AAV. Further, other ITRs, e.g., self-complementary (scAAV) ITRs, may be used. Both single-stranded AAV and self-complementary (sc) AAV are encompassed with the rAAV. The transgene is a nucleic acid coding sequence, heterologous to the vector sequences, which encodes a polypeptide, protein, functional RNA molecule (e.g., miRNA, miRNA inhibitor) or other gene product, of interest. The nucleic acid coding sequence is operatively linked to regulatory components in a manner which permits transgene transcription, translation, and/or expression in a cell of a target tissue. Suitable components of a vector genome are discussed in more detail herein. In one example, a “vector genome” contains, at a minimum, from 5′ to 3′, a vector-specific sequence, a nucleic acid sequence encoding anti-FcRn antibody operably linked to regulatory control sequences (which direct their expression in a target cell), where the vector-specific sequence may be a terminal repeat sequence which specifically packages the vector genome into a viral vector capsid or envelope protein. For example, AAV inverted terminal repeats are utilized for packaging into AAV and certain other parvovirus capsids.

In one aspect, provided is an expression cassette comprising an engineered nucleic acid sequence encoding a nucleic acid sequence (transgene) encoding a desired gene product, and a regulatory sequence which directs expression thereof. In one embodiment, provided is an expression cassette comprising an engineered nucleic acid sequence as described herein which encodes a functional gene product, and a regulatory sequence which directs expression thereof.

The expression cassettes may contain any suitable transgene for delivery to a patient. Particularly suitable are expression cassettes which are to be delivered via the viral vector systemically. Examples of useful genes, coding sequences and gene products are provided below in the section relating to methods of use.

As used herein, the term “expression” or “gene expression” refers to the process by which information from a gene is used in the synthesis of a functional gene product. The gene product may be a protein, a peptide, or a nucleic acid polymer (such as a RNA, a DNA or a PNA).

As used herein, the term “regulatory sequence”, or “expression control sequence” refers to nucleic acid sequences, such as initiator sequences, enhancer sequences, and promoter sequences, which induce, repress, or otherwise control the transcription of protein encoding nucleic acid sequences to which they are operably linked.

The term “exogenous” as used to describe a nucleic acid sequence or protein means that the nucleic acid or protein does not naturally occur in the position in which it exists in a chromosome, or host cell. An exogenous nucleic acid sequence also refers to a sequence derived from and inserted into the same host cell or subject, but which is present in a non-natural state, e.g., a different copy number, or under the control of different regulatory elements.

The term “heterologous” as used to describe a nucleic acid sequence or protein means that the nucleic acid or protein was derived from a different organism or a different species of the same organism than the host cell or subject in which it is expressed. The term “heterologous” when used with reference to a protein or a nucleic acid in a plasmid, expression cassette, or vector (e.g., rAAV), indicates that the protein or the nucleic acid is present with another sequence or subsequence which with which the protein or nucleic acid in question is not found in the same relationship to each other in nature.

In one embodiment, the expression cassette is designed for expression and secretion in a human subject. In one embodiment, the expression cassette is designed for expression in muscle. In one embodiment, the expression cassette is designed for expression in liver.

In certain embodiments, a constitutive promoter may be selected. In one embodiment, the promoter is human cytomegalovirus (CMV) or a chicken β-actin promoter. A variety of chicken beta-actin promoters have been described alone, or in combination with various enhancer elements (e.g., CB7 is a chicken beta-actin promoter with cytomegalovirus enhancer elements; a CAG promoter, which includes the promoter, the first exon and first intron of chicken beta actin, and the splice acceptor of the rabbit beta-globin gene; a CBh promoter, S J Gray et al, Hu Gene Ther, 2011 September; 22(9): 1143-1153). Alternatively, other constitutive promoters may be selected.

Other suitable promoters may include, e.g., a tissue-specific promoter or an inducible/regulatory promoter. Preferably, such promoters are of human origin.

Examples of liver-specific promoters may include, e.g., thyroid hormone-binding globulin (TBG), albumin, Miyatake et al., (1997) J. Virol., 71:5124-32; hepatitis B virus core promoter, Sandig et al., (1996) Gene Ther., 3:1002-9; or human alpha 1-antitrypsin, phosphoenolpyruvate carboxykinase (PECK), or alpha-fetoprotein (AFP), Arbuthnot et al., (1996) Hum. Gene Ther., 7:1503-14). Examples of muscle-specific promoters may include, e.g., the muscle creatine kinase (MCK) promoter and truncated forms thereof. See, e.g., B. Wang, et al, Gene Therapy volume 15, pages 1489-1499 (2008). See, also, muscle-specific transcriptional cis-regulatory modules (CRMs), such as those described S. Sarcare, et al, (January 2019) Nat Commun. 2019; 10: 492.

Alternatively, a regulatable promoter may be selected. See, e.g., WO 2017/106244 which describes different regulatable expression systems and the rapamycin/rapalog inducible system described, e.g., in WO 2007/126798, U.S. Pat. No. 6,506,379, and WO 2011/126808B2, incorporated by reference herein.

In one embodiment, the regulatory sequence further comprises an enhancer. In one embodiment, the regulatory sequence comprises one enhancer. In another embodiment, the regulatory sequence contains two or more expression enhancers. These enhancers may be the same or may be different. For example, an enhancer may include an Alpha mic/bik enhancer or a CMV enhancer. This enhancer may be present in two copies which are located adjacent to one another. Alternatively, the dual copies of the enhancer may be separated by one or more sequences.

In one embodiment, the regulatory sequence further comprises an intron. In a further embodiment, the intron is a chicken beta-actin intron. Other suitable introns include those known in the art may by a human β-globulin intron, and/or a commercially available Promega® intron, and those described in WO 2011/126808.

In one embodiment, the regulatory sequence further comprises a Polyadenylation signal (polyA). In a further embodiment, the polyA is a rabbit globin poly A. See, e.g., WO 2014/151341. Alternatively, another polyA, e.g., a human growth hormone (hGH) polyadenylation sequence, an SV40 polyA, a thymidine kinase (TK) or a synthetic polyA may be included in an expression cassette.

It should be understood that the compositions in the expression cassette described herein are intended to be applied to other compositions, regiments, aspects, embodiments and methods described across the Specification.

### 3. Vector

In one aspect, provided herein is a vector comprising an engineered nucleic acid sequence encoding a functional human gene product and a regulatory sequence which direct expression thereof in a target cell. In certain embodiments, combinations of these vectors are used.

A “vector” as used herein is a biological or chemical moiety comprising a nucleic acid sequence which can be introduced into an appropriate target cell for replication or expression of said nucleic acid sequence. Examples of a vector includes but not limited to a recombinant virus, a plasmid, Lipoplexes, a Polymersome, Polyplexes, a dendrimer, a cell penetrating peptide (CPP) conjugate, a magnetic particle, or a nanoparticle. In one embodiment, a vector is a nucleic acid molecule into which an exogenous or heterologous or engineered nucleic acid encoding a functional gene product(s) may be inserted, which can then be introduced into an appropriate target cell. Such vectors preferably have one or more origin of replication, and one or more site into which the recombinant DNA can be inserted. Vectors often have means by which cells with vectors can be selected from those without, e.g., they encode drug resistance genes. Common vectors include plasmids, viral genomes, and “artificial chromosomes”. Conventional methods of generation, production, characterization, or quantification of the vectors are available to one of skill in the art.

In one embodiment, the vector is a non-viral plasmid that comprises an expression cassette described thereof, e.g., “naked DNA”, “naked plasmid DNA”, RNA, and mRNA; coupled with various compositions and nano particles, including, e.g., micelles, liposomes, cationic lipid-nucleic acid compositions, poly-glycan compositions and other polymers, lipid and/or cholesterol-based-nucleic acid conjugates, and other constructs such as are described herein. See, e.g., X. Su et al, Mol. Pharmaceutics, 2011, 8 (3), pp 774-787; web publication: Mar. 21, 2011; WO2013/182683, WO 2010/053572 and WO 2012/170930, all of which are incorporated herein by reference.

In certain embodiments, the vector described herein is a “replication-defective virus” or a “viral vector” which refers to a synthetic or artificial viral particle in which an expression cassette containing a nucleic acid sequence encoding a functional gene product(s) is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”-containing only the nucleic acid sequence encoding the gene product flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

Suitable viral vectors may include any suitable delivery vector, such as, e.g., a recombinant adenovirus, a recombinant lentivirus, a recombinant bocavirus, a recombinant adeno-associated virus (rAAV), or another recombinant parvovirus (e.g., bocavirus or hybrid AAV/bocavirus), a retroviral vector, adenoviral vector, poxviral vector (e.g., vaccinia viral vector, such as modified vaccinia ankara (MVA)), or alphaviral vector). In certain embodiments, the viral vector is a recombinant AAV for delivery of a gene product to a patient in need thereof.

As used herein, a packaging cell line is used for production of a in vector (e.g., a recombinant AAV). A host cell may be a prokaryotic or eukaryotic cell (e.g., human, insect, or yeast) that contains exogenous or heterologous DNA that has been introduced into the cell by any means, e.g., electroporation, calcium phosphate precipitation, microinjection, transformation, viral infection, transfection, liposome delivery, membrane fusion techniques, high velocity DNA-coated pellets, viral infection and protoplast fusion. Examples of host cells may include, but are not limited to an isolated cell, a cell culture, an Escherichia coli cell, a yeast cell, a human cell, a non-human cell, a mammalian cell, a non-mammalian cell, an insect cell, an HEK-293 cell, a liver cell, a kidney cell, a cell of the central nervous system, a neuron, a glial cell, or a stem cell.

As used herein, the term “target cell” refers to any target cell in which expression of the functional gene product is desired. In certain embodiments, the term “target cell” is intended to reference the cells of the subject being treated. Examples of target cells may include, but are not limited to, a liver cell, skeletal muscle cell, heart cells, etc. Other examples of target cells are described herein.

It should be understood that the compositions in the vector described herein are intended to be applied to other compositions, regiments, aspects, embodiments and methods described across the Specification.

Adeno-Associated Virus (AAV)

In one aspect, provided herein is a recombinant AAV (rAAV) comprising an AAV capsid and a vector genome packaged therein. In certain embodiments, the vector genome comprises an AAV 5′ inverted terminal repeat (ITR), an engineered nucleic acid sequence encoding a gene product as described herein, a regulatory sequence which direct expression of a gene product in a target cell, and an AAV 3′ ITR. The vector genome comprises an AAV 5′ inverted terminal repeat (ITR), an engineered nucleic acid sequence encoding a gene product as described herein, a regulatory sequence which direct expression of the gene product a target cell, and an AAV 3′ ITR. In certain embodiments, the regulatory sequence comprises a tissue-specific promoter (e.g., muscle or liver). In certain embodiments, the regulatory sequence further comprises an enhancer. In one embodiment, the regulatory sequence further comprises an intron. In one embodiment, the regulatory sequence further comprises a poly A. In one embodiment, the AAV capsid is an AAV1 capsid. In certain embodiments, the AAV capsid is an AAV8 capsid. In certain embodiments, the AAV capsid is an AAV9 capsid. In certain embodiments, the AAV capsid is an AAVhu68 capsid. In certain embodiments, the AAV capsid is anAAVrh91 capsid.

In one embodiment, the regulatory sequence is as described above. In one embodiment, the vector genome comprises an AAV 5′ inverted terminal repeat (ITR), an expression cassette as described herein, and an AAV 3′ ITR.

The ITRs are the genetic elements responsible for the replication and packaging of the genome during vector production and are the only viral cis elements required to generate rAAV. In one embodiment, the ITRs are from an AAV different than that supplying a capsid. In a preferred embodiment, the ITR sequences from AAV2, or the deleted version thereof (AITR), which may be used for convenience and to accelerate regulatory approval. However, ITRs from other AAV sources may be selected. Where the source of the ITRs is from AAV2 and the AAV capsid is from another AAV source, the resulting vector may be termed pseudotyped. Typically, AAV vector genome comprises an AAV 5′ ITR, the nucleic acid sequences encoding the gene product(s) and any regulatory sequences, and an AAV 3′ ITR. However, other configurations of these elements may be suitable. In one embodiment, a self-complementary AAV is provided. A shortened version of the 5′ ITR, termed AITR, has been described in which the D-sequence and terminal resolution site (trs) are deleted. In certain embodiments, the vector genome includes a shortened AAV2 ITR of 130 base pairs, wherein the external “a” elements is deleted. The shortened ITR is reverted back to the wild type length of 145 base pairs during vector DNA amplification using the internal A element as a template. In other embodiments, the full-length AAV 5′ and 3′ ITRs are used.

The term “AAV” as used herein refers to naturally occurring adeno-associated viruses, adeno-associated viruses available to one of skill in the art and/or in light of the composition(s) and method(s) described herein, as well as artificial AAVs. An adeno-associated virus (AAV) viral vector is an AAV Dnase-resistant particle having an AAV protein capsid into which is packaged expression cassette flanked by AAV inverted terminal repeat sequences (ITRs) for delivery to target cells. An AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. Various AAVs may be selected as sources for capsids of AAV viral vectors as identified above. In one embodiment, the AAV capsid is an AAV9 capsid or variant thereof. In certain embodiments, the capsid protein is designated by a number or a combination of numbers and letters following the term “AAV” in the name of the rAAV vector. Unless otherwise specified, the AAV capsid, ITRs, and other selected AAV components described herein, may be readily selected from among any AAV, including, without limitation, the AAVs identified as AAV1, AAV2, AAV3, AAV4, AAV5, AAV6, AAV7, AAV8, AAV9, AAVrh10, AAVhu37, AAVrh32.33, AAV8 bp, AAV7M8 and AAVAnc80, AAV1, AAV2, AAV3, AAV4, AAV5, AAV6, AAV7, AAV8, AAV9.47, AAV9(hu14), AAV10, AAV11, AAV12, AAVrh8, AAVrh74, AAV-DJ8, AAV-DJ, AAVhu68, without limitation, See, e.g., WO2019/168961 and WO 2019/169004 (AAV Vectors; Deamidation); WO 2019/169004 (novel AAV capsids); US Published Patent Application No. 2007-0036760-A1; US Published Patent Application No. 2009-0197338-A1; EP 1310571. See also, WO 2003/042397 (AAV7 and other simian AAV), U.S. Pat. Nos. 7,790,449 and 7,282,199 (AAV8), WO 2005/033321 and U.S. Pat. No. 7,906,111 (AAV9), and WO 2006/110689, and WO 2003/042397 (rh.10), and, WO 2005/033321, which are incorporated herein by reference. Other suitable AAVs may include, without limitation, AAVrh90, AAVrh91, AAVrh92, AAVrh93, AAVrh91.93. See, e.g., WO 2020/223232 A1 (AAV rh.90), WO 2020/223231 A1 (AAV rh.91), and WO 2020/223236 A1 (AAV rh.92, AAV rh.93, AAV rh.91.93), which are incorporated herein by reference in its entirety. Other suitable AAV include AAV3B variants which are described in PCT/US20/56511, filed Oct. 20, 2020 (claiming the benefit of U.S. Provisional Patent Application No. 62/924,112, filed Jan. 31, 2020 and U.S. Provisional Patent Application No. 63/025,753, filed May 18, 2020), describing AAV3B.AR2.01, AAV3B.AR2.02, AAV3B.AR2.03, AAV3B.AR2.04 (SEQ ID NO: 8), AAV3B.AR2.05, AAV3B.AR2.06, AAV3B.AR2.07, AAV3B.AR2.08, AAV3B.AR2.10, AAV3B.AR2.11, AAV3B.AR2.12, AAV3B.AR2.13, AAV3B.AR2.14, AAV3B.AR2.15, AAV3B.AR2.16, or AAV3B.AR2.17, which are incorporated herein by reference. These documents also describe other AAV which may be selected for generating AAV and are incorporated by reference. Among the AAVs isolated or engineered from human or non-human primates (NHP) and well characterized, human AAV2 is the first AAV that was developed as a gene transfer vector; it has been widely used for efficient gene transfer experiments in different target tissues and animal models.

As used herein, relating to AAV, the term “variant” means any AAV sequence which is derived from a known AAV sequence, including those with a conservative amino acid replacement, and those sharing at least 70%, at least 75%, at least 80%, at least 85%, at least 90%, at least 95%, at least 97%, at least 99% or greater sequence identity over the amino acid or nucleic acid sequence. In another embodiment, the AAV capsid includes variants which may include up to about 10% variation from any described or known AAV capsid sequence. That is, the AAV capsid shares about 90% identity to about 99.9% identity, about 95% to about 99% identity or about 97% to about 98% identity to an AAV capsid provided herein and/or known in the art. In one embodiment, the AAV capsid shares at least 95% identity with an AAV capsid. When determining the percent identity of an AAV capsid, the comparison may be made over any of the variable proteins (e.g., vp1, vp2, or vp3).

The ITRs or other AAV components may be readily isolated or engineered using techniques available to those of skill in the art from an AAV. Such AAV may be isolated, engineered, or obtained from academic, commercial, or public sources (e.g., the American Type Culture Collection, Manassas, Va.). Alternatively, the AAV sequences may be engineered through synthetic or other suitable means by reference to published sequences such as are available in the literature or in databases such as, e.g., GenBank, PubMed, or the like. AAV viruses may be engineered by conventional molecular biology techniques, making it possible to optimize these particles for cell specific delivery of nucleic acid sequences, for minimizing immunogenicity, for tuning stability and particle lifetime, for efficient degradation, for accurate delivery to the nucleus, etc.

As used herein, the terms “rAAV” and “artificial AAV” used interchangeably, mean, without limitation, a AAV comprising a capsid protein and a vector genome packaged therein, wherein the vector genome comprising a nucleic acid heterologous to the AAV. In one embodiment, the capsid protein is a non-naturally occurring capsid. Such an artificial capsid may be generated by any suitable technique, using a selected AAV sequence (e.g., a fragment of a vp1 capsid protein) in combination with heterologous sequences which may be obtained from a different selected AAV, non-contiguous portions of the same AAV, from a non-AAV viral source, or from a non-viral source. An artificial AAV may be, without limitation, a pseudotyped AAV, a chimeric AAV capsid, a recombinant AAV capsid, or a “humanized” AAV capsid. Pseudotyped vectors, wherein the capsid of one AAV is replaced with a heterologous capsid protein, are useful in the invention. In one embodiment, AAV2/5 and AAV2/8 are exemplary pseudotyped vectors. The selected genetic element may be delivered by any suitable method, including transfection, electroporation, liposome delivery, membrane fusion techniques, high velocity DNA-coated pellets, viral infection and protoplast fusion. The methods used to make such constructs are known to those with skill in nucleic acid manipulation and include genetic engineering, recombinant engineering, and synthetic techniques. See, e.g., Green and Sambrook, Molecular Cloning: A Laboratory Manual, Cold Spring Harbor Press, Cold Spring Harbor, N.Y. (2012).

Methods of generating the capsid, coding sequences therefore, and methods for production of rAAV viral vectors have been described. See, e.g., Gao, et al, Proc. Natl. Acad. Sci. U.S.A. 100 (10), 6081-6086 (2003) and US 2013/0045186A1.

In one embodiment, the rAAV as described herein is a self-complementary AAV. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

In certain embodiments, the rAAV described herein is nuclease-resistant. Such nuclease may be a single nuclease, or mixtures of nucleases, and may be endonucleases or exonucleases. A nuclease-resistant rAAV indicates that the AAV capsid has fully assembled and protects these packaged genomic sequences from degradation (digestion) during nuclease incubation steps designed to remove contaminating nucleic acids which may be present from the production process. In many instances, the rAAV described herein is DNase resistant.

The recombinant adeno-associated virus (AAV) described herein may be generated using techniques which are known. See, e.g., WO 2003/042397; WO 2005/033321, WO 2006/110689; U.S. Pat. No. 7,588,772 B2. Such a method involves culturing a host cell which contains a nucleic acid sequence encoding an AAV capsid; a functional rep gene; an expression cassette as described herein flanked by AAV inverted terminal repeats (ITRs); and sufficient helper functions to permit packaging of the expression cassette into the AAV capsid protein. Also provided herein is the host cell which contains a nucleic acid sequence encoding an AAV capsid; a functional rep gene; a vector genome as described; and sufficient helper functions to permit packaging of the vector genome into the AAV capsid protein. In one embodiment, the host cell is a HEK 293 cell. These methods are described in more detail in WO2017160360 A2, which is incorporated by reference herein.

Other methods of producing rAAV available to one of skill in the art may be utilized. Suitable methods may include without limitation, baculovirus expression system or production via yeast. See, e.g., Robert M. Kotin, Large-scale recombinant adeno-associated virus production. Hum Mol Genet. 2011 Apr. 15; 20(R1): R2-R6. Published online 2011 Apr. 29. doi: 10.1093/hmg/ddr141; Aucoin M G et al., Production of adeno-associated viral vectors in insect cells using triple infection: optimization of baculovirus concentration ratios. Biotechnol Bioeng. 2006 Dec. 20; 95(6):1081-92; SAMI S. THAKUR, Production of Recombinant Adeno-associated viral vectors in yeast. Thesis presented to the Graduate School of the University of Florida, 2012; Kondratov O et al. Direct Head-to-Head Evaluation of Recombinant Adeno-associated Viral Vectors Manufactured in Human versus Insect Cells, Mol Ther. 2017 Aug. 10. pii: 51525-0016(17)30362-3. doi: 10.1016/j.ymthe.2017.08.003. [Epub ahead of print]; Mietzsch M et al, OneBac 2.0: Sf9 Cell Lines for Production of AAV1, AAV2, and AAV8 Vectors with Minimal Encapsidation of Foreign DNA. Hum Gene Ther Methods. 2017 February; 28(1):15-22. doi: 10.1089/hgtb.2016.164; Li L et al. Production and characterization of novel recombinant adeno-associated virus replicative-form genomes: a eukaryotic source of DNA for gene transfer. pLoS One. 2013 Aug. 1; 8(8):e69879. doi: 10.1371/journal.pone.0069879. Print 2013; Galibert L et al, Latest developments in the large-scale production of adeno-associated virus vectors in insect cells toward the treatment of neuromuscular diseases. J Invertebr Pathol. 2011 July; 107 Suppl:S80-93. doi: 10.1016/j.jip.2011.05.008; and Kotin R M, Large-scale recombinant adeno-associated virus production. Hum Mol Genet. 2011 Apr. 15; 20(R1):R2-6. doi: 10.1093/hmg/ddr141. Epub 2011 Apr. 29.

A two-step affinity chromatography purification at high salt concentration followed by anion exchange resin chromatography are used to purify the vector drug product and to remove empty capsids. These methods are described in more detail in WO 2017/160360 entitled “Scalable Purification Method for AAV9”, which is incorporated by reference herein. In brief, the method for separating rAAV9 particles having packaged genomic sequences from genome-deficient AAV9 intermediates involves subjecting a suspension comprising recombinant AAV9 viral particles and AAV 9 capsid intermediates to fast performance liquid chromatography, wherein the AAV9 viral particles and AAV9 intermediates are bound to a strong anion exchange resin equilibrated at a pH of 10.2, and subjected to a salt gradient while monitoring eluate for ultraviolet absorbance at about 260 and about 280. Although less optimal for rAAV9, the pH may be in the range of about 10.0 to 10.4. In this method, the AAV9 full capsids are collected from a fraction which is eluted when the ratio of A260/A280 reaches an inflection point. In one example, for the Affinity Chromatography step, the diafiltered product may be applied to a Capture Select™ Poros-AAV2/9 affinity resin (Life Technologies) that efficiently captures the AAV2/9 serotype. Under these ionic conditions, a significant percentage of residual cellular DNA and proteins flow through the column, while AAV particles are efficiently captured.

Conventional methods for characterization or quantification of rAAV are available to one of skill in the art. To calculate empty and full particle content, VP3 band volumes for a selected sample (e.g., in examples herein an iodixanol gradient-purified preparation where # of GC=# of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and ×100 gives the percentage of empty particles. Generally, methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Viral. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e., SYPRO stain. In one embodiment, the concentration of AAV vector genomes (vg) in column fractions can be measured by quantitative real time PCR (Q-PCR). Samples are diluted and digested with dNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used.

In one aspect, an optimized q-PCR method is used which utilizes a broad spectrum serine protease, e.g., proteinase K (such as is commercially available from Qiagen). More particularly, the optimized qPCR genome titer assay is similar to a standard assay, except that after the dNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2-fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000-fold) and subjected to TaqMan analysis as described in the standard assay.

Additionally, or alternatively, droplet digital PCR (ddPCR) may be used. For example, methods for determining single-stranded and self-complementary AAV vector genome titers by ddPCR have been described. See, e.g., M. Lock et al, Hu Gene Therapy Methods, Hum Gene Ther Methods. 2014 April; 25(2):115-25. doi: 10.1089/hgtb.2013.131. Epub 2014 Feb. 14.

Methods for determining the ratio among vp1, vp2 and vp3 of capsid protein are also available. See, e.g., Vamseedhar Rayaprolu et al, Comparative Analysis of Adeno-Associated Virus Capsid Stability and Dynamics, J Virol. 2013 December; 87(24): 13150-13160; Buller R M, Rose J A. 1978. Characterization of adenovirus-associated virus-induced polypeptides in KB cells. J. Virol. 25:331-338; and Rose J A, Maizel J V, Inman J K, Shatkin A J. 1971. Structural proteins of adenovirus-associated viruses. J. Virol. 8:766-770.

As used herein, a “stock” of rAAV refers to a population of rAAV. Despite heterogeneity in their capsid proteins due to deamidation, rAAV in a stock are expected to share an identical vector genome. A stock can include rAAV having capsids with, for example, heterogeneous deamidation patterns characteristic of the selected AAV capsid proteins and a selected production system. The stock may be produced from a single production system or pooled from multiple runs of the production system. A variety of production systems, including but not limited to those described herein, may be selected.

It should be understood that the compositions in the rAAV described herein are intended to be applied to other compositions, regimens, aspects, embodiments and methods described across the Specification.

### 5. Pharmaceutical Composition

In one aspect, provided herein is a pharmaceutical composition comprising a vector as described herein in a formulation buffer. In certain embodiments, the pharmaceutical composition comprising the vector further comprises an anti-FcRn ligand, e.g., anti-FcRn antibody as described herein. In certain embodiments, one or more the anti-FcRn ligands are formulated and delivered separately from the vector. In one embodiment, provided is a pharmaceutical composition comprising a rAAV as described herein in a formulation buffer. In certain embodiments, provided is a pharmaceutical composition comprising a receptor-targeted nanoparticles comprising encapsulated nucleic acid sequence encoding the anti-FcRn ligand (e.g., anti-FcRn antibody) as described herein in a formulation buffer.

In one embodiment, the formulation further comprises a surfactant, preservative, excipients, and/or buffer dissolved in the aqueous suspending liquid. In one embodiment, the buffer is PBS. Suitably, the formulation is adjusted to a physiologically acceptable pH, e.g., in the range of pH 6 to 8; for intravenous delivery, a pH of 6.8 to about 7.2 may be desired. However, other pHs within the broadest ranges and these subranges may be selected for other route of delivery.

A suitable surfactant, or combination of surfactants, may be selected from among non-ionic surfactants that are nontoxic. In one embodiment, a difunctional block copolymer surfactant terminating in primary hydroxyl groups is selected, e.g., such as Pluronic® F68 [BASF], also known as Poloxamer 188, which has a neutral pH, has an average molecular weight of 8400. Other surfactants and other Poloxamers may be selected, i.e., nonionic triblock copolymers composed of a central hydrophobic chain of polyoxypropylene (poly (propylene oxide)) flanked by two hydrophilic chains of polyoxyethylene (poly (ethylene oxide)), SOLUTOL HS 15 (Macrogol-15 Hydroxystearate), LABRASOL (Caprylocaproyl macrogol glycerides), polyoxy 10 oleyl ether, TWEEN (polyoxyethylene sorbitan fatty acid esters), ethanol and polyethylene glycol. In one embodiment, the formulation contains a poloxamer. These copolymers are commonly named with the letter “P” (for poloxamer) followed by three digits: the first two digits×100 give the approximate molecular mass of the polyoxypropylene core, and the last digit×10 gives the percentage polyoxyethylene content. In one embodiment Poloxamer 188 is selected. The surfactant may be present in an amount up to about 0.0005% to about 0.001% of the suspension.

Additionally provided is a pharmaceutical composition comprising a pharmaceutically acceptable carrier and a vector comprising a nucleic acid sequence encoding a functional gene product as described herein. As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. Delivery vehicles such as liposomes, nanocapsules, microparticles, microspheres, lipid particles, vesicles, and the like, may be used for the introduction of the compositions of the present invention into suitable host cells. In particular, the rAAV vector may be formulated for delivery either encapsulated in a lipid particle, a liposome, a vesicle, a nanosphere, or a nanoparticle or the like. In one embodiment, a therapeutically effective amount of said vector is included in the pharmaceutical composition. The selection of the carrier is not a limitation of the present invention. Other conventional pharmaceutically acceptable carrier, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The phrase “pharmaceutically-acceptable” refers to molecular entities and compositions that do not produce an allergic or similar untoward reaction when administered to a host.

As used herein, the term “dosage” or “amount” can refer to the total dosage or amount delivered to the subject in the course of treatment, or the dosage or amount delivered in a single unit (or multiple unit or split dosage) administration.

Also, the replication-defective virus compositions can be formulated in dosage units to contain an amount of replication-defective virus that is in the range of about 1.0×10⁹ GC to about 1.0×10¹⁶ GC (to treat an average subject of 70 kg in body weight) including all integers or fractional amounts within the range, and preferably 1.0×10¹² GC to 1.0×10¹⁴ GC for a human patient. In one embodiment, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another embodiment, the compositions are formulated to contain at least 1×10¹⁰, 2×10¹⁰ 3×10¹⁰ 4×10¹⁰, 5×10¹⁰, 6×10¹⁰, 7×10¹⁰, 8×10¹⁰, or 9×10¹⁰ GC per dose including all integers or fractional amounts within the range. In another embodiment, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another embodiment, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another embodiment, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another embodiment, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another embodiment, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In one embodiment, for human application the dose can range from 1×10¹⁰ to about 1×10¹² GC per dose including all integers or fractional amounts within the range.

In one embodiment, the pharmaceutical composition comprising a rAAV as described herein is administrable at a dose of about 1×10⁹ GC per gram of brain mass to about 1×10¹⁴ GC per gram of brain mass.

The aqueous suspension or pharmaceutical compositions described herein are designed for delivery to subjects in need thereof by any suitable route or a combination of different routes. In one embodiment, the pharmaceutical composition is formulated for delivery via intracerebroventricular (ICV), intrathecal (IT), or intracisternal injection. In one embodiment, the compositions described herein are designed for delivery to subjects in need thereof by intravenous injection. Alternatively, other routes of administration may be selected (e.g., oral, inhalation, intranasal, intratracheal, intraarterial, intraocular, intramuscular, and other parenteral routes).

In certain embodiments, the aqueous suspension or the pharmaceutical composition is used in preparing a medicament. In certain embodiments, uses of the same are for reducing levels of neutralizing antibodies to a vector (e.g., parental AAV capsid source) in a patient in a need thereof are provided.

It should be understood that the compositions in the pharmaceutical composition described herein are intended to be applied to other compositions, regiments, aspects, embodiments and methods described across the Specification.

### 6. Method of Treatment

In one embodiment, a combination regimen for treating a patient with neutralizing antibodies to a viral vector is provided. The regimen comprises administering a vector in combination with a ligand which inhibits binding of human FcRn and pre-existing patient neutralizing antibodies (e.g, IgG). The FcRn ligand (i.e., anti-FcRn) is as described herein. In certain embodiments, the ligand is an anti-FcRn antibody construct, and the vector is a recombinant viral vector. The vector may be recombinant adeno-associated virus (rAAV) or another viral vector as described herein (e.g., a recombinant adenovirus, a recombinant herpes simplex virus, or a recombinant lentivirus, a recombinant retroviral vector, a recombinant poxviral vector (e.g., vaccinia viral vector, such as Modified Vaccinia Ankara (MVA)), or alphaviral vector)) and the patient selected may have neutralizing antibodies to the vector (e.g., parental AAV capsid source). In certain embodiments, the patient has pre-existing anti-AAV8 antibodies, and the combination comprises co-administration of an anti-FcRn ligand and an rAAV8 vector or a cross-reactive vector. In certain embodiments, the patient has pre-existing anti-AAV2 antibodies, and the combination comprises co-administration of an anti-FcRn ligand and an rAAV2 vector or a cross-reactive vector (e.g., having a capsid from an AAV2 variant or a different AAV Clade B AAV)). In certain embodiments, the patient has pre-existing anti-Adenovirus type 5 and is co-administered an anti-FcRn ligand and a recombinant adenovirus having a type 5 capsid. Other virus types and subtypes may be selected in view of the teachings of this specification.

In certain embodiments, the patient may be naïve to any therapeutic treatment with a selected viral vector and may have pre-existing immunity due to prior infections with a wild-type virus. In other embodiments, the patient may have neutralizing antibodies as a result of a prior treatment or vaccine. In certain embodiments, the patient may have neutralizing antibodies of about 1:1 to about 1:20, or in excess of 1:2, in excess of 1:5, in excess of 1:10, in excess of 1:20, in excess of 1:50, in excess of 1:100, in excess of 1:200, in excess of 1:300 or higher. In certain embodiments, a patient has neutralizing antibodies in the range of 1:1 to 1:200, or 1:5 to 1:100, or 1:2 to 1:20, or 1:5 to 1:50, or 1:5 to 1:20. In certain embodiments, a patient has neutralizing antibodies in the range of greater than 1 to about 5. In certain embodiments, a patient has neutralizing antibodies in the range of about 1 to about 20. In certain embodiments, a patient has neutralizing antibodies in the range of about 1 to about 40. In certain embodiments, a patient has neutralizing antibodies in the range of about 1 to about 80. In certain embodiments, a patient receives a single anti-FcRn ligand (e.g., anti-FcRn antibody) as the sole agent to modulate FcRn-IgG binding and to permit effective vector delivery. In other embodiments, a patient may receive a combination of one or more anti-FcRn ligands and a second component (e.g., an Fc receptor down-regulator (e.g., interferon gamma), an IgG enzyme, or another suitable component). Such combinations may be particularly desirable for patients having particularly high neutralizing antibody levels (e.g., in excess of 1:200). In certain embodiments, the compositions comprising anti-FcRn ligands, and the regimens and co-administration are utilized during systemic delivery of viral vectors. However, the invention is not so limited, as described in more detail herein.

In certain embodiments, an anti-FcRn ligand(s) (e.g., antibodies) is administered to a patient having neutralizing antibodies prior to and, optionally, concurrently with a selected viral vector. In certain embodiments, continued expression of an anti-FcRn ligand post-administration of the gene therapy vector may desired on a short-term (transient basis), e.g., until such time as the viral vector clears from the patient. In certain embodiments, persistent expression of an anti-FcRn ligand may be desired. Optionally, in this embodiment, the ligand may be delivered via a viral vector, including, e.g., in the viral vector expressing the therapeutic transgene. However, this embodiment is not desirable where the therapeutic gene being delivered is an antibody or antibody construct or another construct comprising an IgG chain. In such embodiments, where an antibody construct having an IgG chain is being delivered via a viral vector to a patient having pre-existing immunity, the anti-FcRn ligand is delivered or dosed transiently so that the amount of anti-FcRn ligand in the circulation is cleared from the sera before effective levels of vector-mediated transgene product are expressed.

In certain embodiments, the FcRn ligand is delivered one to seven days prior to administration of the vector (e.g., rAAV). In certain embodiments, the FcRn ligand is delivered daily. In certain embodiments, the FcRn ligand (e.g., immunoglobulin construct(s)) is delivered on the same day as the vector is administered. In certain embodiments, the FcRn ligand (e g, immunoglobulin construct(s)) is delivered at least one day to four weeks post-rAAV administration. In certain embodiments, the ligand is delivered for four weeks to 6 months post-rAAV administration. In certain embodiments, the ligand is dosed via a different route of administration than the rAAV. In certain embodiments, the ligand is dosed orally, intravenously, or intraperitoneally.

In certain embodiments, the patient has pre-existing neutralizing antibodies as a result of WT infection (e.g., with WT AAV) has not previously received vector-based gene therapy treatment prior to the delivery of the vector in combination with the anti-FcRn immunoglobulin construct. In certain embodiments, the patient has a neutralizing titer greater than 1:5 as determined in an in vitro assay. In certain embodiments, the patient has previously received gene therapy prior to the delivery of the vector (e.g., rAAV) in combination with the anti-FcRn immunoglobulin construct.

In certain embodiments, the method is part of a regimen which further comprises co-administering one or more of: (a) a steroid or combination of steroids and/or (b) an IgG-cleaving enzyme, (c) an inhibitor of Fc-IgE binding; (d) an inhibitor of Fc-IgM binding; (e) an inhibitor of Fc-IgA binding; and/or (f) gamma interferon.

The efficacy of the compositions and regimens provided herein may be determined, e.g., by measuring NAb titers. Additionally or alternatively, the efficacy of the compositions and regimens may be determined using assays for detecting transgene expression post-vector mediated delivery. Such assays may be the same as those used to detect transgene expression in patients not testing positive neutralizing antibodies, or a predetermined threshold of neutralizing antibodies.

Examples of suitable transgenes for delivery include, e.g., those associated with familial hypercholesterolemia (e.g., VLDLr, LDLr, ApoE, PCSK9), muscular dystrophy, cystic fibrosis, and rare or orphan diseases. Examples of such rare disease may include spinal muscular atrophy (SMA), Huntingdon's Disease, Rett Syndrome (e.g., methyl-CpG-binding protein 2 (MeCP2); UniProtKB-P51608), Amyotrophic Lateral Sclerosis (ALS), Duchenne Type Muscular dystrophy, Friedrichs Ataxia (e.g., frataxin), progranulin (PRGN) (associated with non-Alzheimer's cerebral degenerations, including, frontotemporal dementia (FTD), progressive non-fluent aphasia (PNFA) and semantic dementia), among others. Other useful gene products include, carbamoyl synthetase I, ornithine transcarbamylase (OTC), arginosuccinate synthetase, arginosuccinate lyase (ASL) for treatment of arginosuccinate lyase deficiency, arginase, fumarylacetate hydrolase, phenylalanine hydroxylase, alpha-1 antitrypsin, rhesus alpha-fetoprotein (AFP), rhesus chorionic gonadotrophin (CG), glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin gene product [e.g., a mini- or micro-dystrophin]. Still other useful gene products include enzymes such as may be useful in enzyme replacement therapy, which is useful in a variety of conditions resulting from deficient activity of enzyme. For example, enzymes that contain mannose-6-phosphate may be utilized in therapies for lysosomal storage diseases (e.g., a suitable gene includes that encoding β-glucuronidase (GUSB)). Example of suitable transgene for delivery may include human frataxin delivered in an AAV vector as described, e.g., PCT/US20/66167, Dec. 18, 2020, U.S. Provisional Patent Application No. 62/950,834, filed Dec. 19, 2019, and U.S. Provisional Application No. 63/136,059 filed on Jan. 11, 2021 which are incorporated herein by reference. Another example of suitable transgene for delivery may include human acid-α-glucosidase (GAA) delivered in an AAV vector as described, e.g., PCT/US20/30493, Apr. 30, 2020, now published as WO2020/223362A1, PCT/US20/30484, Apr. 20, 2020, now published as WO 2020/223356 A1, U.S. Provisional Patent Application No. 62/840,911, filed Apr. 30, 2019, US Provisional Application No. 62/913,401, filed Oct. 10, 2019, U.S. Provisional Patent Application No. 63/024,941, filed May 14, 2020, and U.S. Provisional Patent Application No. 63/109,677, filed Nov. 4, 2020 which are incorporated herein by reference. Also, another example of suitable transgene for delivery may include human α-L-iduronidase (IDUA) delivered in an AAV vector as described, e.g., PCT/US2014/025509, Mar. 13, 2014, now published as WO 2014/151341, and U.S. Provisional Patent Application No. 61/788,724, filed Mar. 15, 2013 which are incorporated herein by reference.

Further illustrative genes which may be delivered via the vector (e.g., rAAV) include, without limitation, glucose-6-phosphatase, associated with glycogen storage disease or deficiency type 1A (GSD1), phosphoenolpyruvate-carboxykinase (PEPCK), associated with PEPCK deficiency; cyclin-dependent kinase-like 5 (CDKLS), also known as serine/threonine kinase 9 (STK9) associated with seizures and severe neurodevelopmental impairment; galactose-1 phosphate uridyl transferase, associated with galactosemia; phenylalanine hydroxylase, associated with phenylketonuria (PKU); branched chain alpha-ketoacid dehydrogenase, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; ornithine transcarbamylase (OTC), associated with ornithine transcarbamylase deficiency; argininosuccinic acid synthetase (ASS1), associated with citrullinemia; lecithin-cholesterol acyltransferase (LCAT) deficiency; a methylmalonic acidemia (MMA); Niemann-Pick disease, type C1); propionic academia (PA); low density lipoprotein receptor (LDLR) protein, associated with familial hypercholesterolemia (FH); UDP-glucouronosyltransferase, associated with Crigler-Najjar disease; adenosine deaminase, associated with severe combined immunodeficiency disease; hypoxanthine guanine phosphoribosyl transferase, associated with Gout and Lesch-Nyan syndrome; biotimidase, associated with biotimidase deficiency; alpha-galactosidase A (a-Gal A) associated with Fabry disease); ATP7B associated with Wilson's Disease; beta-glucocerebrosidase, associated with Gaucher disease type 2 and 3; peroxisome membrane protein 70 kDa, associated with Zellweger syndrome; arylsulfatase A (ARSA) associated with metachromatic leukodystrophy, galactocerebrosidase (GALC) enzyme associated with Krabbe disease, alpha-glucosidase (GAA) associated with Pompe disease; sphingomyelinase (SMPD1) gene associated with Nieman Pick disease type A; argininosuccinate synthase associated with adult onset type II citrullinemia (CTLN2); carbamoyl-phosphate synthase 1 (CPS1) associated with urea cycle disorders; survival motor neuron (SMN) protein, associated with spinal muscular atrophy; ceramidase associated with Farber lipogranulomatosis; b-hexosaminidase associated with GM2 gangliosidosis and Tay-Sachs and Sandhoff diseases; aspartylglucosaminidase associated with aspartyl-glucosaminuria; a-fucosidase associated with fucosidosis; α-mannosidase associated with alpha-mannosidosis; porphobilinogen deaminase, associated with acute intermittent porphyria (AIP); alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease; the beta adrenergic receptor, anti-sense to, or a mutant form of, phospholamban, the sarco(endo)plasmic reticulum adenosine triphosphatase-2 (SERCA2), and the cardiac adenylyl cyclase for the treatment of congestive heart failure; a tumor suppressor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin or GLP-1 for the treatment of diabetes. Additional genes and diseases of interest include, e.g., dystonin gene related diseases such as Hereditary Sensory and Autonomic Neuropathy Type VI (the DST gene encodes dystonin; dual AAV vectors may be required due to the size of the protein (˜7570 aa); SCN9A related diseases, in which loss of function mutants cause inability to feel pain and gain of function mutants cause pain conditions, such as erythromelagia. Another condition is Charcot-Marie-Tooth type 1F and 2E due to mutations in the NEFL gene (neurofilament light chain). characterized by a progressive peripheral motor and sensory neuropathy with variable clinical and electrophysiologic expression. In certain embodiments, the vectors described herein may be used in treatment of mucopolysaccaridoses (MPS) disorders. Such vectors may contain carry a nucleic acid sequence encoding α-L-iduronidase (IDUA) for treating MPS I (Hurler, Hurler-Scheie and Scheie syndromes); a nucleic acid sequence encoding iduronate-2-sulfatase (IDS) for treating MPS II (Hunter syndrome); a nucleic acid sequence encoding sulfamidase (SGSH) for treating MPSIII A, B, C, and D (Sanfilippo syndrome); a nucleic acid sequence encoding N-acetylgalactosamine-6-sulfate sulfatase (GALNS) for treating MPS IV A and B (Morquio syndrome); a nucleic acid sequence encoding arylsulfatase B (ARSB) for treating MPS VI (Maroteaux-Lamy syndrome); a nucleic acid sequence encoding hyaluronidase for treating MPSI IX (hyaluronidase deficiency) and a nucleic acid sequence encoding beta-glucuronidase for treating MPS VII (Sly syndrome). See, e.g., www.orpha.net/consor/cgi-bin/Disease_Search_List.php; rarediseases.info.nih.gov/diseases.

Nucleic acid sequences encoding receptors for cholesterol regulation and/or lipid modulation may also be selected, including the low density lipoprotein (LDL) receptor, high density lipoprotein (HDL) receptor, the very low density lipoprotein (VLDL) receptor, and scavenger receptors. Other suitable gene products may include members of the steroid hormone receptor superfamily including glucocorticoid receptors and estrogen receptors, Vitamin D receptors and other nuclear receptors. In addition, useful gene products include transcription factors such as jun, fos, max, mad, serum response factor (SRF), AP-1, AP2, myb, MyoD and myogenin, ETS-box containing proteins, TFE3, E2F, ATF1, ATF2, ATF3, ATF4, ZF5, NFAT, CREB, HNF-4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

Examples of other suitable genes may include, e.g., hormones and growth and differentiation factors including, without limitation, insulin, glucagon, glucagon-like peptide-1 (GLP1), growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO) (including, e.g., human, canine or feline epo), connective tissue growth factor (CTGF), neutrophic factors including, e.g., basic fibroblast growth factor (bFGF), acidic fibroblast growth factor (aFGF), epidermal growth factor (EGF), platelet-derived growth factor (PDGF), insulin growth factors I and II (IGF-I and IGF-II), any one of the transforming growth factor α superfamily, including TGFα, activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Other useful transgene products include proteins that regulate the immune system including, without limitation, cytokines and lymphokines such as thrombopoietin (TPO), interleukins (IL) IL-1 through IL-36 (including, e.g., human interleukins IL-1, IL-1α, IL-1β, IL-2, IL-3, IL-4, IL-6, IL-8, IL-12, IL-11, IL-12, IL-13, IL-18, IL-31, IL-35), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand. Gene products produced by the immune system are also useful in the invention. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. For example, in certain embodiments, the rAAV antibodies may be designed to delivery canine or feline antibodies, e.g., such as anti-IgE, anti-IL31, anti-CD20, anti-NGF, anti-GnRH. Useful gene products also include complement regulatory proteins such as complement regulatory proteins, membrane cofactor protein (MCP), decay accelerating factor (DAF), CR1, CF2, CD59, and C1 esterase inhibitor (C1-INH). Still other useful gene products include any one of the receptors for the hormones, growth factors, cytokines, lymphokines, regulatory proteins and immune system proteins.

Examples of suitable transgenes useful in treatment of one or more neurodegenerative disorders. Such disorders may include, without limitation, transmissible spongiform encephalopathies (e.g., Creutzfeld-Jacob disease), Duchenne muscular dystrophy (DMD), myotubular myopathy and other myopathies, Parkinson's disease, amyotrophic lateral sclerosis (ALS), multiple sclerosis, Alzheimer's Disease, Huntington disease, Canavan's disease, traumatic brain injury, spinal cord injury (ATI335, anti-nogol by Novartis), migraine (ALD403 by Alder Biopharmaceuticals; LY2951742 by Eli; RN307 by Labrys Biologics), lysosomal storage diseases, stroke, and infectious disease affecting the central nervous system. Examples of lysosomal storage disease include, e.g., Gaucher disease, Fabry disease, Niemann-Pick disease, Hunter syndrome, glycogen storage disease II (Pompe disease), or Tay-Sachs disease. For certain of these conditions, e.g., DMD and myopathies, the compositions provided herein are useful in reducing or eliminating axonopathy associated with high doses of expression cassettes (e.g., carried by a viral vector) for transduction or invention of skeletal and cardiac muscle.

Suitable transgenes may also include antibodies expressed in vivo. Certain embodiments permit continued delivery (or expression) of an anti-FcRn ligand(s) (e.g., antibodies) with therapeutic gene which is delivered via the viral vector. However, this embodiment is not desirable where the therapeutic gene being delivered is an antibody or antibody construct or another construct comprising an IgG chain. In such embodiments, where an antibody construct having an IgG chain is being delivered via a viral vector to a patient having pre-existing immunity, the anti-FcRn ligand is delivered or dosed transiently so that the amount of anti-FcRn ligand in the circulation is cleared from the sera before effective levels of vector-mediated transgene product are expressed.

Still other nucleic acids may encode an immunoglobulin which is directed to leucine rich repeat and immunoglobulin-like domain-containing protein 1 (LINGO-1), which is a functional component of the Nogo receptor and which is associated with essential tremors in patients which multiple sclerosis, Parkinson's Disease or essential tremor. One such commercially available antibody is ocrelizumab (Biogen, BIIB033). See, e.g., U.S. Pat. No. 8,425,910. In one embodiment, the nucleic acid constructs encode immunoglobulin constructs useful for patients with ALS. Examples of suitable antibodies include antibodies against the ALS enzyme superoxide dismutase 1 (SOD1) and variants thereof (e.g., ALS variant G93A, C4F6 SOD1 antibody); MS785, which directed to Derlin-1-binding region); antibodies against neurite outgrowth inhibitor (NOGO-A or Reticulon 4), e.g., GSK1223249, ozanezumab (humanized, GSK, also described as useful for multiple sclerosis). Nucleic acid sequences may be designed or selected which encode immunoglobulins useful in patients having Alzheimer's Disease. Such antibody constructs include, e.g., adumanucab (Biogen), Bapineuzumab (Elan; a humanised mAb directed at the amino terminus of Aβ); Solanezumab Eli Lilly, a humanized mAb against the central part of soluble Aβ); Gantenerumab (Chugai and Hoffmann-La Roche, is a full human mAb directed against both the amino terminus and central portions of Aβ); Crenezumab (Genentech, a humanized mAb that acts on monomeric and conformational epitopes, including oligomeric and protofibrillar forms of Aβ; BAN2401 (Esai Co., Ltd, a humanized immunoglobulin G1 (IgG1) mAb that selectively binds to Aβ protofibrils and is thought to either enhance clearance of Aβ protofibrils and/or to neutralize their toxic effects on neurons in the brain); GSK 933776 (a humanised IgG1 monoclonal antibody directed against the amino terminus of Aβ); AAB-001, AAB-002, AAB-003 (Fc-engineered bapineuzumab); SAR228810 (a humanized mAb directed against protofibrils and low molecular weight Aβ); BIIB037/BART (a full human IgG1 against insoluble fibrillar human Aβ, Biogen Idec), an anti-Aβ antibody such m266, tg2576 (relative specificity for Aβ oligomers) [Brody and Holtzman, Annu Rev Neurosci, 2008; 31: 175-193]. Other antibodies may be targeted to beta-amyloid proteins, Aβ, beta secretase and/or the tau protein. In still other embodiments, an anti-β-amyloid antibody is derived from an IgG4 monoclonal antibodies to target β-amyloid in order to minimize effector functions, or construct other than an scFv which lacks an Fc region is selected in order to avoid amyloid related imaging abnormality (ARIA) and inflammatory response. In certain of these embodiments, the heavy chain variable region and/or the light chain variable region of one or more of the scFv constructs is used in another suitable immunoglobulin construct as provided herein. These scFV and other engineered immunoglobulins may reduce the half-life of the immunoglobulin in the serum, as compared to immunoglobulins containing Fc regions. Reducing the serum concentration of anti-amyloid molecules may further reduce the risk of ARIA, as extremely high levels of anti-amyloid antibodies in serum may destabilize cerebral vessels with a high burden of amyloid plaques, causing vascular permeability. Nucleic acids encoding other immunoglobulin constructs for treatment of patients with Parkinson's disease may be engineered or designed to express constructs, including, e.g., leucine-rich repeat kinase 2, dardarin (LRRK2) antibodies; anti-synuclein and alpha-synuclein antibodies and DJ-1 (PARK7) antibodies. Other antibodies may include, PRX002 (Prothena and Roche) Parkinson's disease and related synucleinopathies. These antibodies, particularly anti-synuclein antibodies may also be useful in treatment of one or more lysosomal storage disease.

One may engineer or select nucleic acid constructs encoding an immunoglobulin construct for treating multiple sclerosis. Such immunoglobulins may include or be derived from antibodies such as natalizumab (a humanized anti-a4-ingrin, iNATA, Tysabri, Biogen Idec and Elan Pharmaceuticals), which was approved in 2006, alemtuzumab (Campath-1H, a humanized anti-CD52), rituximab (rituzin, a chimeric anti-CD20), daclizumab (Zenepax, a humanized anti-CD25), ocrelizumab (humanized, anti-CD20, Roche), ustekinumab (CNTO-1275, a human anti-IL12 p40+IL23p40); anti-LINGO-1, and ch5D12 (a chimeric anti-CD40), and rHIgM22 (a remyelinated monoclonal antibody; Acorda and the Mayo Foundation for Medical Education and Research). Still other anti-a4-integrin antibodies, anti-CD20 antibodies, anti-CD52 antibodies, anti-IL17, anti-CD19, anti-SEMA4D, and anti-CD40 antibodies may be delivered via the AAV vectors as described herein.

Antibodies against various infections of the central nervous system is also contemplated by the present invention. Such infectious diseases may include fungal diseases such as cryptoccocal meningitis, brain abscess, spinal epidural infection caused by, e.g., Cryptococcus neoformans, Coccidioides immitis, order Mucorales, Aspergillus spp, and Candida spp; protozoal, such as toxoplasmosis, malaria, and primary amoebic meningoencephalitis, caused by agents such as, e.g., Toxoplasma gondii, Taenia solium, Plasmodium falciparus, Spirometra mansonoides (sparaganoisis), Echinococcus spp (causing neuro hydatosis), and cerebral amoebiasis; bacterial, such as, e.g., tuberculosis, leprosy, neurosyphilis, bacterial meningitis, lyme disease (Borrelia burgdorferi), Rocky Mountain spotted fever (Rickettsia rickettsia), CNS nocardiosis (Nocardia spp), CNS tuberculosis (Mycobacterium tuberculosis), CNS listeriosis (Listeria monocytogenes), brain abscess, and neuroborreliosis; viral infections, such as, e.g., viral meningitis, Eastern equine encephalitis (EEE), St Louis encepthalitis, West Nile virus and/or encephalitis, rabies, California encephalitis virus, La Crosse encepthalitis, measles encephalitis, poliomyelitis, which may be caused by, e.g., herpes family viruses (HSV), HSV-1, HSV-2 (neonatal herpes simplex encephalitis), varicella zoster virus (VZV), Bickerstaff encephalitis, Epstein-Barr virus (EBV), cytomegalovirus (CMV, such as TCN-202 is in development by Theraclone Sciences), human herpesvirus 6 (HHV-6), B virus (herpesvirus simiae), Flavivirus encephalitis, Japanese encephalitis, Murray valley fever, JC virus (progressive multifocal leukoencephalopathy), Nipah Virus (NiV), measles (subacute sclerosing panencephalitis); and other infections, such as, e.g., subactuate sclerosing panencephalitis, progressive multifocal leukoencephalopathy; human immunodeficiency virus (acquired immunodeficiency syndrome (AIDS)); Streptococcus pyogenes and other β-hemolytic Streptococcus (e.g., Pediatric Autoimmune Neuropsychiatric Disorders Associated with Streptococcal Infection, PANDAS) and/or Syndenham's chorea, and Guillain-Barre syndrome, and prions.

Examples of suitable antibody constructs may include those described, e.g., in WO 2007/012924A2, Jan. 29, 2015, which is incorporated by reference herein.

For example, other nucleic acid sequences may encode anti-prion immunoglobulin constructs. Such immunoglobulins may be directed against major prion protein (PrP, for prion protein or protease-resistant protein, also known as CD230 (cluster of differentiation 230). The amino acid sequence of PrP is provided, e.g., ncbi.nlm.nih.gov/protein/NP_000302, incorporated by reference herein. The protein can exist in multiple isoforms, the normal PrPC, the disease-causing PrPSc, and an isoform located in mitochondria. The misfolded version PrPSc is associated with a variety of cognitive disorders and neurodegenerative diseases such as Creutzfeldt-Jakob disease, bovine spongiform encephalopathy, Gerstmann-Sträussler-Scheinker syndrome, fatal familial insomnia, and kuru.

In certain embodiments, a method for increasing the patient population for which gene therapy is effective is provided. The method comprises co-administering to a patient from a population having a neutralizing antibody titer to a selected viral capsid or a serologically cross-reactive capsid which is greater than 1:5; (a) a recombinant virus having the selected viral capsid and a gene therapy expression cassette packaged therein; and (b) a ligand which specifically binds the neonatal Fc receptor (FcRn) prior to delivery of the gene therapy vector, wherein the ligand blocks the FcRN binding to immunoglobulin G (IgG) and permits effective amounts of the gene therapy product to be expressed in the patient.

In certain embodiments, a method for treating a patient with neutralizing antibodies to a capsid of a recombinant adeno-associated virus (rAAV) is provided. The method comprises administering the rAAV in combination with an anti-neonatal Fc receptor (FcRn) immunoglobulin construct as defined herein, wherein said immunoglobulin construct specifically inhibits FcRn binding to an immunoglobulin G (IgG), suitably without interfering with FcRn-albumin binding.

In certain embodiments, the viral vector (e.g, rAAV) is delivered systemically. In certain embodiments, the rAAV is delivered intravenously, intraperitoneally, intranasally, or via inhalation. In certain embodiments, the rAAV has a capsid selected from AAV1, AAV2, AAV3, AAV5, AAV7, AAV8, AAV9, AAVrh10, AAVhu37. In certain embodiments, the rAAV has an AAVhu68 capsid. In certain embodiments, the rAAV has an AAVrh91 capsid. In certain embodiments, the immunoglobulin construct is a monoclonal antibody nipocalimab (M281) or an immunoglobulin construct which comprises three or more CDRs thereof, or combinations thereof. In certain embodiments, the immunoglobulin construct is selected from rozanolixizumab (UCB7665), IMVT-1401, RVT-1401, HL161, HBM916, ARGX-113 (efgartigimod), SYNT001, SYNT002, ABY-039, or DX-2507, or a derivative of the immunoglobulin construct, or a combination of the immunoglobulin constructs and/or derivates thereof.

In one embodiment, the subject is delivered a therapeutically effective amount of the vectors described herein. As used herein, a “therapeutically effective amount” refers to the amount of the composition comprising the nucleic acid sequence encoding a functional gene which delivers and expresses in the target cells an amount of enzyme sufficient to achieve efficacy. In one embodiment, the dosage of the vector is about 1×10⁹ GC to about 1×10¹² genome copies (GC) per dose. In specific embodiments, the dose of the vector administered to a patient is at least about 1.0×10⁹ GC/kg, about 1.5×10⁹ GC/kg, about 2.0×10⁹ GC/g, about 2.5×10⁹ GC/kg, about 3.0×10⁹ GC/kg, about 3.5×10⁹ GC/kg, about 4.0×10⁹ GC/kg, about 4.5×10⁹ GC/kg, about 5.0×10⁹ GC/kg, about 5.5×10⁹ GC/kg, about 6.0×10⁹ GC/kg, about 6.5×10⁹ GC/kg, about 7.0×10⁹ GC/kg, about 7.5×10⁹ GC/kg, about 8.0×10⁹ GC/kg, about 8.5×10⁹ GC/kg, about 9.0×10⁹ GC/kg, about 9.5×10⁹ GC/kg, about 1.0×10¹⁰ GC/kg, about 1.5×10¹⁰ GC/kg, about 2.0×10¹⁰ GC/kg, about 2.5×10¹⁰ GC/kg, about 3.0×10¹⁰ GC/kg, about 3.5×10¹⁰ GC/kg, about 4.0×10¹⁰ GC/kg, about 4.5×10¹⁰ GC/kg, about 5.0×10¹⁰ GC/kg, about 5.5×10¹⁰ GC/kg, about 6.0×10¹⁰ GC/kg, about 6.5×10¹⁰ GC/kg, about 7.0×10¹⁰ GC/kg, about 7.5×10¹⁰ GC/kg, about 8.0×10¹⁰ GC/kg, about 8.5×10¹⁰ GC/kg, about 9.0×10¹⁰ GC/kg, about 9.5×10¹⁰ GC/kg, about 1.0×10¹¹ GC/kg, about 1.5×10¹¹ GC/kg, about 2.0×10¹¹ GC/kg, about 2.5×10¹¹ GC/kg, about 3.0×10¹¹ GC/kg, about 3.5×10¹¹ GC/kg, about 4.0×10¹¹ GC/kg, about 4.5×10¹¹ GC/kg, about 5.0×10¹¹ GC/kg, about 5.5×10¹¹ GC/kg, about 6.0×10¹¹ GC/kg, about 6.5×10¹¹ GC/kg, about 7.0×10¹¹ GC/kg, about 7.5×10¹¹ GC/kg, about 8.0×10¹¹ GC/kg, about 8.5×10¹¹ GC/kg, about 9.0×10¹¹ GC/kg, about 9.5×10¹¹ GC/kg, about 1.0×10¹² GC/kg, about 1.5×10¹² GC/kg, about 2.0×10¹² GC/kg, about 2.5×10¹² GC/kg, about 3.0×10¹² GC/kg, about 3.5×10¹² GC/kg, about 4.0×10¹² GC/kg, about 4.5×10¹² GC/kg, about 5.0×10¹² GC/kg, about 5.5×10¹² GC/kg, about 6.0×10¹² GC/kg, about 6.5×10¹² GC/kg, about 7.0×10¹² GC/kg, about 7.5×10¹² GC/kg, about 8.0×10¹² GC/kg, about 8.5×10¹² GC/kg, about 9.0×10¹² GC/kg, about 9.5×10¹² GC/kg, about 1.0×10¹³ GC/kg, about 1.5×10¹³ GC/kg, about 2.0×10¹³ GC/kg, about 2.5×10¹³ GC/kg, about 3.0×10¹³ GC/kg, about 3.5×10¹³ GC/kg, about 4.0×10¹³ GC/kg, about 4.5×10¹³ GC/kg, about 5.0×10¹³ GC/kg, about 5.5×10¹³ GC/kg, about 6.0×10¹³ GC/kg, about 6.5×10¹³ GC/kg, about 7.0×10¹³ GC/kg, about 7.5×10¹³ GC/kg, about 8.0×10¹³ GC/kg, about 8.5×10¹³ GC/kg, about 9.0×10¹³ GC/kg, about 9.5×10¹³ GC/kg, or about 1.0×10¹⁴ GC/kg.

In one embodiment, the method further comprises the subject receives an immunosuppressive co-therapy Immunosuppressants for such co-therapy include, but are not limited to, a glucocorticoid, steroids, antimetabolites, T-cell inhibitors, a macrolide (e.g., a rapamycin or rapalog), and cytostatic agents including an alkylating agent, an anti-metabolite, a cytotoxic antibiotic, an antibody, or an agent active on immunophilin. The immune suppressant may include a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor-(CD25-) or CD3-directed antibodies, anti-IL-2 antibodies, ciclosporin, tacrolimus, sirolimus, IFN-β, IFN-γ, an opioid, or TNF-α (tumor necrosis factor-alpha) binding agent.

In certain embodiments, the immunosuppressive therapy may be started 0, 1, 2, 7, or more days prior to the gene therapy administration. Such therapy may involve co-administration of two or more drugs, the (e.g., prednelisone, mycophenolic acid (MMF) and/or sirolimus (i.e., rapamycin)) on the same day. One or more of these drugs may be continued after gene therapy administration, at the same dose or an adjusted dose. Such therapy may be for about 1 week (7 days), about 60 days, or longer, as needed. In certain embodiments, a tacrolimus-free regimen is selected. In one embodiment, the rAAV as described herein is administrated once to the subject in need. In another embodiment, the rAAV is administrated more than once to the subject in need.

It should be understood that the compositions in the method described herein are intended to be applied to other compositions, regiments, aspects, embodiments and methods described across the Specification.

### 7. Kit

In certain embodiments, a kit is provided which includes a concentrated vector suspended in a formulation (optionally frozen), optional dilution buffer, and devices and components required for administration. In one embodiment, the kit provides sufficient buffer to allow for injection. In certain embodiments, the kit provides sufficient buffer to allow for intranasal or aerosolizing administration. Such buffer may allow for about a 1:1 to a 1:5 dilution of the concentrated vector, or more. In other embodiments, higher or lower amounts of buffer or sterile water are included to allow for dose titration and other adjustments by the treating clinician. In still other embodiments, one or more components of the device are included in the kit. Suitable dilution buffer is available, such as, a saline, a phosphate buffered saline (PBS) or a glycerol/PBS.

Optionally, the kit further comprises a composition comprising the anti-FcRn ligand (e.g, immunoglobulin, antibody construct) for delivery.

It should be understood that the compositions in kit described herein are intended to be applied to other compositions, regimens, aspects, embodiments and methods described across the Specification.

As used herein, the phrases “ameliorate a symptom”, “improve a symptom” or any grammatical variants thereof, refer to reversal of a symptoms, symptom or prevention of progression of symptoms associated with the gene product being delivered. In one embodiment, the amelioration or improvement refers to the total number of symptoms in a patient after administration of the described composition(s) or use of the described method, which is reduced by about 5%, about 10%, about 20%, about 30%, about 40%, about 50%, about 60%, about 70%, about 80%, about 90%, about 95% compared to that before the administration or use. In another embodiment, the amelioration or improvement refers to the severity or progression of a symptom after administration of the described composition(s) or use of the described method, which is reduced by about 5%, about 10%, about 20%, about 30%, about 40%, about 50%, about 60%, about 70%, about 80%, about 90%, about 95% compared to that before the administration or use.

Unless otherwise specified, “patient” refers to a male or female human and “subject” refers to ns a male or female human, dogs, and animal models used for clinical research.

In certain embodiments, a combination regimen for treating a gene therapy patient with neutralizing antibodies to the outer capsid or envelope protein of desired viral vector. The regimen comprises co-administering a viral vector carrying an expression cassette comprising a nucleic acid sequence encoding a gene product operably linked to regulatory sequences which direct expression thereof in the target cell, in a therapeutic combination with a ligand which inhibits binding of a human neonatal Fc receptor (FcRn) and an immunoglobulin G (IgG) which is directed against the outer capsid or envelope protein of the viral vector. Suitably, the ligand is directed against FcRn-IgG binding without interfering with FcRn-albumin binding. The ligand may be selected from a peptide, protein, an RNAi sequence, or a small molecule. In certain embodiments, the ligand protein is a monoclonal antibody, an immunoadhesin, a camelid antibody, a Fab fragment, an Fv fragment, or an scFv fragment. The viral vector may be, without limitation, a recombinant adeno-associated virus, a recombinant adenovirus, a recombinant herpes simplex virus, or a recombinant lentivirus.

In certain embodiments, the regimen comprises treatment of the patient with a monoclonal antibody selected from nipocalimab (M281), rozanolixizumab (UCB7665); IMVT-1401, RVT-1401, HL161, HBM916, ARGX-113 (efgartigimod), orilanolimab (SYNT001), SYNT002, ABY-039, or DX-2507, derivatives or combinations thereof.

In certain embodiments, the ligand is nipocalimab (M281) or an immunoglobulin construct which comprises three or more CDRs thereof selected from: (a) heavy chain CDRs of (i) CDR H1, SEQ ID NO: 16 or a sequence at least 99% identical thereto, (ii) CDR H2, SEQ ID NO:18 or a sequence at least 99% identical thereto, and (iii) CDR H3, SEQ ID NO: 20 or a sequence at least 99% identical thereto; or (b) light chain CDRs of: CDR L1, SEQ ID NO: 10 or a sequence at least 99% identical thereto, CDR L2, SEQ ID NO:12 or a sequence at least 99% identical thereto, CDR L3, SEQ ID NO: 14, or a sequence at least 99% identical thereto. In certain embodiments, nipocalimab or the immunoglobulin construct comprises: (a) heavy chain CDRs of (i) CDR H1, SEQ ID NO: 16, (ii) CDR H2, SEQ ID NO:18, and (iii) CDR H3, SEQ ID NO: 20; or (b) light chain CDRs of: CDR L1, SEQ ID NO: 10, CDR L2, SEQ ID NO:12, CDR L3, SEQ ID NO: 14.

In certain embodiments, nucleic acid sequences encoding these ligands, or another selected ligand, are encompassed by the methods and compositions provided herein. The ligand (e.g., anti-FcRn antibody) may be expressed in vivo following administration of a vector comprising nucleic acid sequences encoding the ligand (e.g., anti-FcRn antibody) which are operably linked to regulatory control sequences which direct expression of the ligand.

In certain embodiments, prior to dosing with the combination regimen, the patient has a neutralizing titer greater than 1:5 against the rAAV capsid or a serologically cross-reactive capsid as determined in an in vitro assay.

In certain embodiments, the patient may be treated with the ligand (i.e., ligand delivered) one to seven days prior to administration or delivery of the viral vector, on the same day as the viral vector, and/or for a day, days, weeks, or months (e.g., 10 days to 6 months, or longer, about 2 weeks to 12 weeks, or longer) post-delivery with the viral vector. Optionally, the ligand is delivered daily. In certain embodiments, the ligand is delivered via a different route of administration than the viral vector. The ligand may be delivered orally. The viral vector may be delivered intraperitoneally, intravenously, intramuscularly, intranasally, or intrathecally.

In certain embodiments, prior to treatment the patient is predetermined to have a neutralizing antibody titer to the vector capsid which greater than 1:5 as determined in an in vitro assay. In certain embodiments, the patient has not previously received gene therapy treatment or gene delivery using a viral vector prior to the delivery of the viral vector in combination with the inhibitory ligand such that the patient's pre-existing neutralizing antibodies are a result of wild-type viral vector infection. In certain embodiments, the patient has previously received gene therapy treatment prior to the delivery of the viral vector in combination with the inhibitory ligand.

In certain embodiments, a combination regimen provided herein further comprises co-administering one or more of: (a) a steroid or combination of steroids; and/or (b) an IgG-cleaving enzyme; and (c) an inhibitor of Fc-IgE binding; (d) an inhibitor of Fc-IgM binding; (e) an inhibitor of Fc-IgA binding; and/or (f) gamma interferon.

In certain embodiments, the methods described herein are effective for expanding (increasing) the patient population for which gene therapy is effective. For example, such a method may comprise co-administering to a patient from a population having a neutralizing antibody titer to a selected viral capsid or a serologically cross-reactive capsid which is greater than 1:5: (a) a recombinant virus having the selected viral capsid and a gene therapy expression cassette packaged in the selected viral capsid; and (b) a ligand which specifically binds a neonatal Fc receptor (FcRn) prior to delivery of the gene therapy vector, wherein the ligand blocks binding of the FcRN to immunoglobulin G (IgG), and permits effective amounts of the gene therapy product to be expressed in the patient. In certain embodiments, a method is provided for treating a patient with neutralizing antibodies to a capsid of a recombinant adeno-associated virus (rAAV), the method comprising administering the rAAV in combination with an anti-neonatal Fc receptor (FcRn) immunoglobulin construct, wherein said immunoglobulin construct specifically inhibits FcRn-immunoglobulin G (IgG) binding. In certain embodiments, the rAAV is delivered systemically, e.g., intravenously, intraperitoneally, intranasally, or via inhalation. Any suitable capsid may be selected, but in certain embodiments, the rAAV has a capsid selected from AAV1, AAV2, AAV3, AAV5, AAV7, AAV8, AAV9, AAVrh10, AAVrh91, AAVhu37, AAVhu68.

With regard to the description of these inventions, it is intended that each of the compositions herein described, is useful, in another embodiment, in the methods of the invention. In addition, it is also intended that each of the compositions herein described as useful in the methods, is, in another embodiment, itself an embodiment of the invention.

Unless defined otherwise in this specification, technical and scientific terms used herein have the same meaning as commonly understood by one of ordinary skill in the art to which this invention belongs and by reference to published texts, which provide one skilled in the art with a general guide to many of the terms used in the present application.

A nucleic acid refers to a polymeric form of nucleotides and includes RNA, mRNA, cDNA, genomic DNA, peptide nucleic acid (PNA) and synthetic forms and mixed polymers of the above. A nucleotide refers to a ribonucleotide, deoxynucleotide or a modified form of either type of nucleotide (e.g., a peptide nucleic acid oligomer). The term also includes single- and double-stranded forms of DNA. The skilled man will appreciate that functional variants of these nucleic acid molecules are also intended to be a part of the present invention. Functional variants are nucleic acid sequences that can be directly translated, using the standard genetic code, to provide an amino acid sequence identical to that translated from the parental nucleic acid molecules.

Methods are known and have been described previously (e.g., WO 96/09378). A sequence is considered engineered if at least one non-preferred codon as compared to a wild type sequence is replaced by a codon that is more preferred. Herein, a non-preferred codon is a codon that is used less frequently in an organism than another codon coding for the same amino acid, and a codon that is more preferred is a codon that is used more frequently in an organism than a non-preferred codon. The frequency of codon usage for a specific organism can be found in codon frequency tables, such as in www.kazusa.jp/codon. Preferably more than one non-preferred codon, preferably most or all non-preferred codons, are replaced by codons that are more preferred. Preferably the most frequently used codons in an organism are used in an engineered sequence. Replacement by preferred codons generally leads to higher expression. It will also be understood by a skilled person that numerous different nucleic acid molecules can encode the same polypeptide as a result of the degeneracy of the genetic code. It is also understood that skilled persons may, using routine techniques, make nucleotide substitutions that do not affect the amino acid sequence encoded by the nucleic acid molecules to reflect the codon usage of any particular host organism in which the polypeptides are to be expressed. Therefore, unless otherwise specified, a “nucleic acid sequence encoding an amino acid sequence” includes all nucleotide sequences that are degenerate versions of each other and that encode the same amino acid sequence. Nucleic acid sequences can be cloned using routine molecular biology techniques, or generated de novo by DNA synthesis, which can be performed using routine procedures by service companies having business in the field of DNA synthesis and/or molecular cloning (e.g., GeneArt, GenScript, Life Technologies, Eurofins).

The term “percent (%) identity”, “sequence identity”, “percent sequence identity”, or “percent identical” in the context of nucleic acid sequences refers to the residues in the two sequences which are the same when aligned for correspondence. The length of sequence identity comparison may be over the full-length of the genome, the full-length of a gene coding sequence, or a fragment of at least about 500 to 5000 nucleotides, is desired. However, identity among smaller fragments, e.g., of at least about nine nucleotides, usually at least about 20 to 24 nucleotides, at least about 28 to 32 nucleotides, at least about 36 or more nucleotides, may also be desired.

Multiple sequence alignment programs are also available for nucleic acid sequences. Examples of such programs include, “Clustal Omega”, “Clustal W”, “CAP Sequence Assembly”, “BLAST”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference.

Percent identity may be readily determined for amino acid sequences over the full-length of a protein, polypeptide, about 32 amino acids, about 330 amino acids, or a peptide fragment thereof or the corresponding nucleic acid sequence coding sequences. A suitable amino acid fragment may be at least about 8 amino acids in length, and may be up to about 700 amino acids. Generally, when referring to “identity”, “homology”, or “similarity” between two different sequences, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence.

Unless otherwise specified by an upper range, it will be understood that a percentage of identity is a minimum level of identity and encompasses all higher levels of identity up to 100% identity to the reference sequence. Unless otherwise specified, it will be understood that a percentage of identity is a minimum level of identity and encompasses all higher levels of identity up to 100% identity to the reference sequence. For example, “95% identity” and “at least 95% identity” may be used interchangeably and include 95, 96, 97, 98, 99 up to 100% identity to the referenced sequence, and all fractions therebetween.

Unless otherwise specified, numerical values will be understood to be subject to conventional mathematic rounding rules.

Identity may be determined by preparing an alignment of the sequences and through the use of a variety of algorithms and/or computer programs known in the art or commercially available (e.g., BLAST, ExPASy; Clustal Omega; FASTA; using, e.g., Needleman-Wunsch algorithm, Smith-Waterman algorithm). Alignments are performed using any of a variety of publicly or commercially available Multiple Sequence Alignment Programs. Sequence alignment programs are available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

The aqueous suspension or pharmaceutical compositions described herein are designed for delivery to subjects in need thereof by any suitable route or a combination of different routes.

As used herein, the terms “intrathecal delivery” or “intrathecal administration” refer to a route of administration for drugs via an injection into the spinal canal, more specifically into the subarachnoid space so that it reaches the cerebrospinal fluid (CSF). Intrathecal delivery may include lumbar puncture, intraventricular, suboccipital/intracisternal, and/or C1-2 puncture. For example, material may be introduced for diffusion throughout the subarachnoid space by means of lumbar puncture. In another example, injection may be into the cisterna magna. Intracisternal delivery may increase vector diffusion and/or reduce toxicity and inflammation caused by the administration. See, e.g., Christian Hinderer et al, Widespread gene transfer in the central nervous system of cynomolgus macaques following delivery of AAV9 into the cisterna magna, Mol Ther Methods Clin Dev. 2014; 1: 14051. Published online 2014 Dec. 10. doi: 10.1038/mtm.2014.51.

As used herein, the terms “intracisternal delivery” or “intracisternal administration” refer to a route of administration for drugs directly into the cerebrospinal fluid of the brain ventricles or within the cisterna magna cerebellomedularis, more specifically via a suboccipital puncture or by direct injection into the cisterna magna or via permanently positioned tube.

“Comprising” is a term meaning inclusive of other components or method steps. When “comprising” is used, it is to be understood that related embodiments include descriptions using the “consisting of” terminology, which excludes other components or method steps, and “consisting essentially of” terminology, which excludes any components or method steps that substantially change the nature of the embodiment or invention. It should be understood that while various embodiments in the specification are presented using “comprising” language, under various circumstances, a related embodiment is also described using “consisting of” or “consisting essentially of” language.

It is to be noted that the term “a” or “an”, refers to one or more, for example, “a vector”, is understood to represent one or more rAAV(s) or another specified vector. As such, the terms “a” (or “an”), “one or more,” and “at least one” is used interchangeably herein.

As used herein, the term “about” means a variability of plus or minus 10% from the reference given, unless otherwise specified.

In certain instances, the term “E+#” or the term “e+#” is used to reference an exponent. For example, “5E10” or “5e10” is 5×10¹⁰. These terms may be used interchangeably.

### 8. EXAMPLES

These examples are provided for the purpose of illustration only and the invention should in no way be construed as being limited to these examples but rather should be construed to encompass any and all variations that become evident as a result of the teaching provided herein.

The inventors have developed a strategy to treat subjects with pre-existing neutralizing antibodies with an AAV vector. Using a single dose of a monoclonal antibody targeting the neonatal Fc receptor (FcRn), pre-existing neutralizing antibodies are temporarily reduced up to 10-fold, allowing effective AAV administration.

In mice treated with human IgG, a single FcRN mAb dose resulted in a 10-fold decrease in antibody titer and allowed for successful AAV-mediated liver transduction.

### Example 1. Effects of Blocking FcRn on NAb Titer and AAV Transduction in Mice

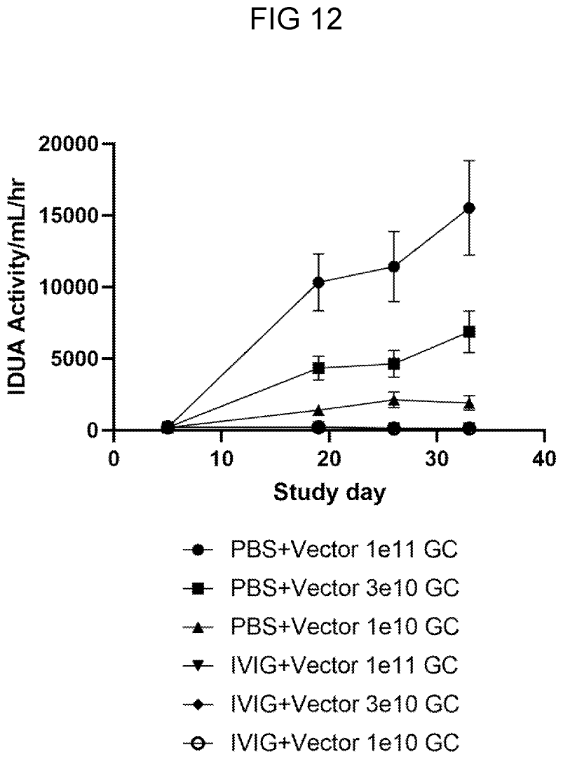

In this study we tested whether anti-FcRn antibody can reduce levels of anti-AAV NAb and thereby enhance an AAV-mediated gene transduction following intravenous (i.v.) administration of AAV in mice. In this study, we observed that administration of M281 monoclonal antibody (mAb) reverts the human NAb-mediated blockage of liver-targeted gene transduction following the intravenous vector administration in humanized FcRn transgenic mice pre-treated with pooled human IgG. FIG. 12 shows results of diminished TT1 activity levels in mice co-treated with IVIG at the time of AAV8.TT1 vector administration.

Table 1 below, shows a summarized study design to examine the effects of blocking FcRn on NAb titer and AAV transduction in mice. In this study, we used human FcRn transgenic mice (i.e., SCID-hFcRnTg32 mice (JAX: 018441)), which provide a longer serum half-life of human immunoglobulin G (hIgG) by the expression of human FcRn, the target of M281 antibody. M281 is an hIgG1 antibody, comprising a heavy chain M281-HC of SEQ ID NO: 8 and a light chain of SEQ ID NO: 7. At the start of the study, day 0, mice were injected intravenously at a dose of 0.5 g/kg with Privigen (IVIG). Privigen had AAV8 neutralizing antibodies (NAb) at a ratio 1:320 in 0.1 g/ml solution. On day 1, mice were injected intraperitoneally with either M281 at a dose of 30 mg/kg (Group 2) or PBS in control group (Group 1). On day 16, mice were administered AAV8.TBG.TT1 at a dose of 4×10¹² GC/kg. AAV8.TBG.TT1 is a vector with an AAV8 capsid and a vector genome encoding a Test Transgene 1 (i.e., TT1) transgene. Serum levels of hIgG/Nab titers were measured as a readout of the study.

TABLE 1

| | | Group 1 | Group 2 | |
|---|---|---|---|---|
| Day | Treatment | N = 8 | N = 5 | Samples |
| −7 | | | | Serum |
| 0 | IVIG (i.v.) 0.5 g/kg | x | x | Serum |
| 1 | M281 (i.p.) 30 mg/kg | PBS | PBS | Serum |
| 8 | | | | Serum |
| 16 | AAV8.TBG.TT1 | x | x | Serum |
| | (i.v. 4e12 GC/kg) | | | |
| 44 | | | | Serum |
| 72 | | | | Necropsy (serum, liver, heart) |

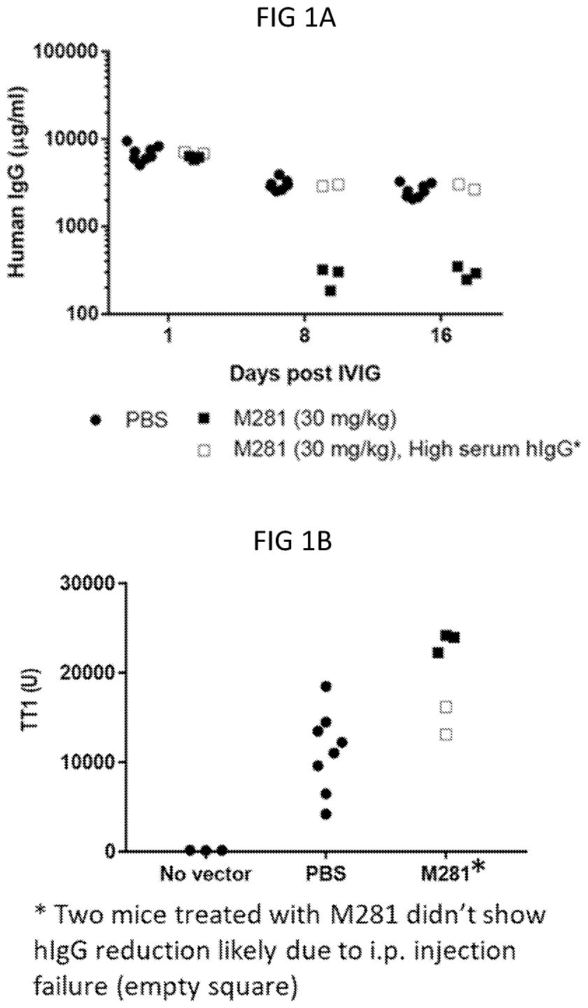

By day 16, levels of hIgG including NAb were reduced to about 50%. FIGS. 1A and 1B show administration of M281 mAb decreased levels of hIgG and improved AAV transduction in livers of hFcRn mice when pre-treated with IVIG. FIG. 1A shows levels of serum human IgG at days 1 to 16 post IVIG pre-treatment. FIG. 1B shows levels, represented in units (U), of transgene activity at 28 days post AAV transduction. Squares represent mice that received the M281 intravenous injection. Filled in squares represent mice in which IgG level reduction was observed. Empty squares represent mice in which the serum levels of IgG were high in comparison and are correlated with the PBS-treated group. The two mice that were treated with M281 (empty squares) and not showing hIgG reduction is likely due to intraperitoneal injection failure.

We next examined the effect of M281 on transgene transduction by intravenous AAV administration at a higher dose in hFcRn Tg mice, which were pre-treated with IVIG. Table 2 below shows a summarized study design for examining the effect of blocking FcRn and correlation between NAb titer and AAV8.TBG.TT1 transduction at higher dose of 1×10¹¹ GC per mouse.

TABLE 2

| Group Designation | 1 | 2 | 3 |
|---|---|---|---|
| N/Group | 5 | 5 | 5 |
| Description | No IVIG | IVIG + PBS | IVIG + M281 |
| Genotype | hFcRnTg32 | hFcRnTg32 | hFcRnTg32 |
| IVIG (iv) − Day 0 | Vehicle control | 1 g/kg | 1 g/kg |
| M281 (ip) − 6 h and | — | Vehicle control | 30 mg/kg |
| 24 h | | | |
| Vector Dose (iv) | 1e11 GC/mouse | 1e11 GC/mouse | 1e11 GC/mouse |

| | |
|---|---|
| Duration, Readouts | 35 day study |
| | Serum NAb, BAb, and IgG (in-life) and Vector |
| | genome biodistribution post necropsy |

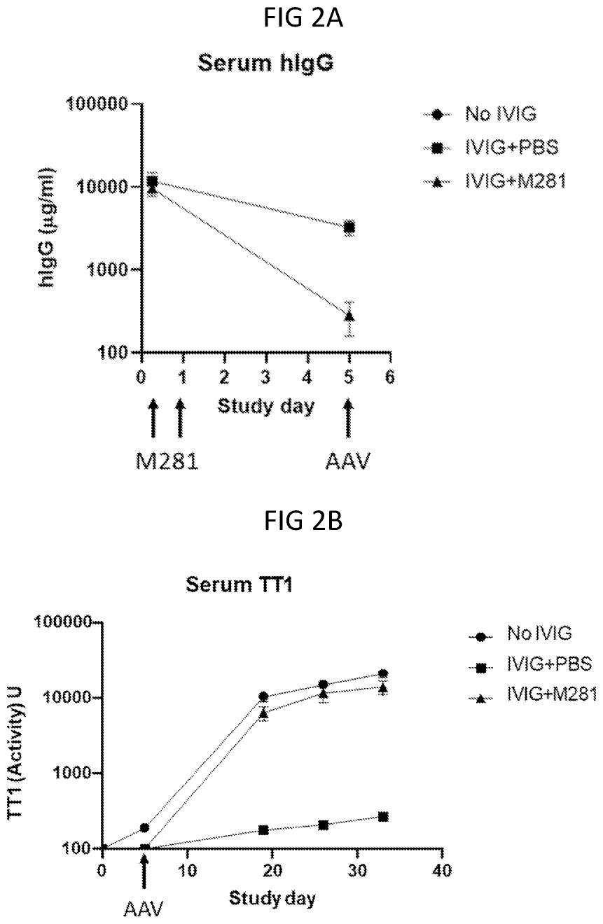

In this experiment, humanized FcRn transgenic scid mice were used. For the experimental group, mice treated with intravenous pooled human IgG at 1 g/kg at day 0 received intraperitoneal M281 injections at 6 and 24 hours (30 mg/kg for each time point) post human IgG. A control group did not receive human IgG or M281 and another control group received human IgG but M281 mAb. At day 5, all mice received intravenous AAV8.TBG.TT1 vector (1×10″ GC/mouse) to examine the liver-targeted gene transduction at day 19, 26, and 33 by serum TT1 activity. Serum levels of NAb (neutralizing binding antibodies), BAb (non-neutralizing binding antibodies) and hIgG (human IgG), and vector genome biodistribution were measured as a readout of the study. Human IgG ELISA showed a significant decrease of human IgG in mice treated with M281 mAb to less than 10% of those treated with human IgG but M281 mAb at day 5 post human IgG (FIG. 2A). Human IgG completely diminished serum TT1 activity, when no M281 mAb was administrated. In contrast, mice without human IgG showed significant increase of serum TT1 activity suggesting that NAb derived from pooled human IgG blocked liver-targeted gene transduction by IV vector. The >90% reduction of human IgG by M281 mAb restored the liver transduction to more than 60% of that in mice untreated with human IgG. These results prove that the long serum half-life of anti-AAV NAb depends on FcRn receptor in vivo, and the blockage of the receptor by M281 mAb reduces circulating IgG with NAb. FIG. 2A shows levels of serum human IgG (hIgG) post pre-treatment with IVIG. Administration of M281 and AAV are indicated by arrows. Serum hIgG levels were decreased in mice treated with M281 (Group 3) on day 5 of study, results of which are summarized in Table 3 below. FIG. 2B shows vector biodistribution levels in serum at day 0 to day 35 of study. TT1 activity levels in serum were similar to those in mice of Control Group 1, which were not pre-treated with IVIG. Inhibition of FcRn by M281 reduced IVIG-derived NAb together with total hIgG and permitted liver gene transduction with intravenous AAV8.

TABLE 3

| Group | AAV8 NAb at day 5 |
|---|---|
| No IVIG | <1:5 |
| MG + PBS | 1:10 |
| MG + M281 | <1:5 |

We have confirmed the efficacy of anti-FcRn antibody M281 for reduction of levels of human IgG and enhancement of AAV mediated gene delivery in the human FcRn transgenic mouse model when infused with pooled human IgG (IVIG).

### Example 2. Effects of Blocking FcRn on NAb Titer and AAV Transduction in Non-Human Primates (NHPs)

In this study, we tested the effect of M281 on pre-existing NAb in NHPs and heart gene transduction following intravenous delivery of an AAVhu68 vector (AAVhu68 capsid and vector genome having a CB7 promoter and Test Transgene 2 transgene (i.e., TT2)). In this study, we observed that administration of M281 mAb transiently reduces pre-existing NAb titer and enhances gene transduction with intravenous vector administration in non-human primates with endogenous pre-existing NAb.

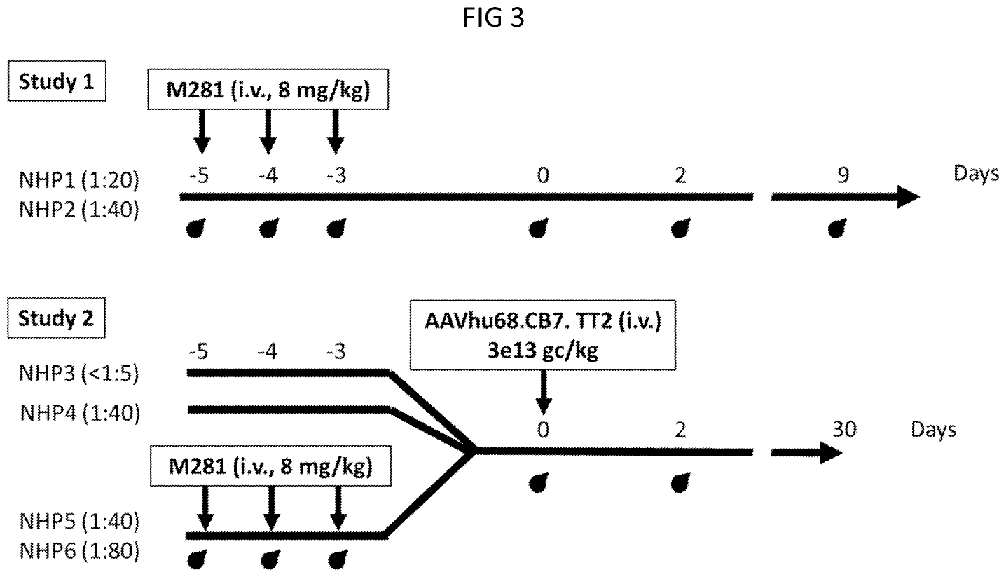

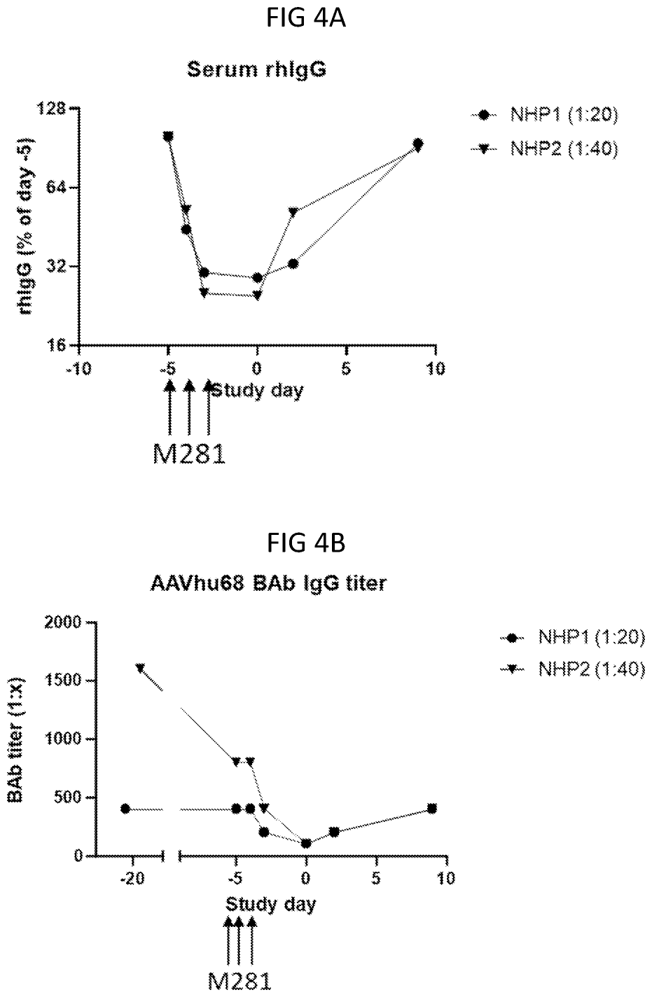

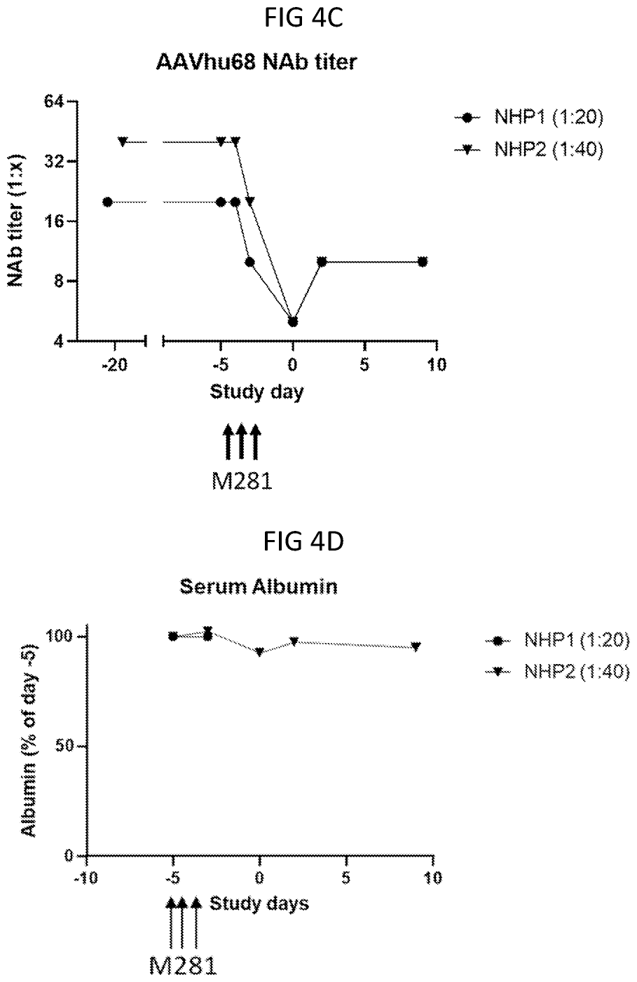

FIG. 3 shows study design to examine the effects of blocking FcRn on NAb titer and AAV transduction in NHPs. In our study we used rhesus macaques (NHPs) with a measured pre-existing NAb titer of 1:80, 1:40, 1:20 and/or <1:5, where indicated. NHPs were dosed with M281 intravenously at a dose of 8 mg/kg on day 5, 4, and 3 (−5, −4, and −3) prior to AAV injection (day 0). On day 0, NHPs were intravenously injected with an AAVhu68 vector at 3×10¹³ GC/kg. In initial Study 1 (as indicated in FIG. 3), two rhesus macaques with pre-existing AAVhu68 NAb titer 1:20 and 1:40, respectively, were treated with intravenous M281 mAb at 8 mg/kg for 3 consecutive days (days −5, −4, and −3) to examine the effect of FcRn blockage by M281 on pre-existing NAb titer in non-human primate. As shown in FIG. 4B, AAVhu68 NAb titer reduced to half at day −3 and then reached to 1:5 at day 0 in both animals. NAb titer showed a small increase to 1:10 at day 2 and remained at 1:10 until day 9. These results clearly indicate that M281 mAb cross-reacts to rhesus FcRn and reduces endogenous rhesus IgG together with NAb. The M281 dosing protocol was indicated to be effective for reducing NAb titer to 1:5 for NHPs with up to 1:40 pre-existing NAb titers. FIGS. 4A to 4D show M281 infusion reduced pre-existing NAb titer and IgG in NHPs. FIG. 4A shows levels of serum rhesus macaque IgG (rhIgG), plotted as percent of day −5, where days for administration of M281 are indicated by arrows on graph. FIG. 4B shows AAVhu68-non-neutralizing binding antibody (BAb) titers, where days for administration of M281 are indicated by arrows on graph. FIG. 4C shows AAVhu68 neutralizing binding antibody (NAb) titers, where days for administration of M281 are indicated by arrows on graph. FIG. 4D shows levels of serum albumin plotted as percent of day −5, wherein M281 administration is indicated by arrows on graph.

Next, in study 2 (as indicated in FIG. 3) we treated 2 rhesus macaques with AAVhu68 NAb titer 1:40 and 1:80, respectively, with M281 mAb and then dosed vector intravenously to test whether the NAb reduction by M281 mAb has positive impact on gene transduction in the context of intravenous AAV gene therapy. In this study, AAVhu68 vector expressing TT2 (3×10¹³ GC/kg) was intravenously administered at day 0 following the 3-day intravenous injections of 8 mg/kg M281 at days −5, −4, and −3. 1 NHP with NAb<1:5 and another NHP with NAb 1:40 were used as NAb-negative and NAb-positive controls, respectively, which received only IV vector but M281 mAb pre-treatment. NAb was reduced and reached to 1:5 at day 0 in NHPs treated with M281 together with serum total IgG and AAV-binding antibody. Upon vector injection, there was a robust increase of NAb titer that reached 1:1280 in 7 days post-AAV in these animals. While the increase was 1-dilution lower than NAb-positive control monkey which showed 1:2560 at day 7, it was much higher compared with NAb-negative control monkey which showed 1:160 at day 7. Vector genome biodistribution was analyzed in the heart, liver, spleen, and skeletal muscle after necropsy at day 30. Vector Genome copy number was diminished in the heart, liver, and skeletal muscle from NAb-positive control monkey compared with NAb-negative monkey. That was improved in the liver and skeletal muscle from M281-treated monkeys. For heart, there was a small improvement in the copy number in one of M281-treated monkeys which had 1:40 NAb at the baseline. Vector genome tends to accumulate to the spleen in NAb-positive monkeys due to the antibody-mediated immune response

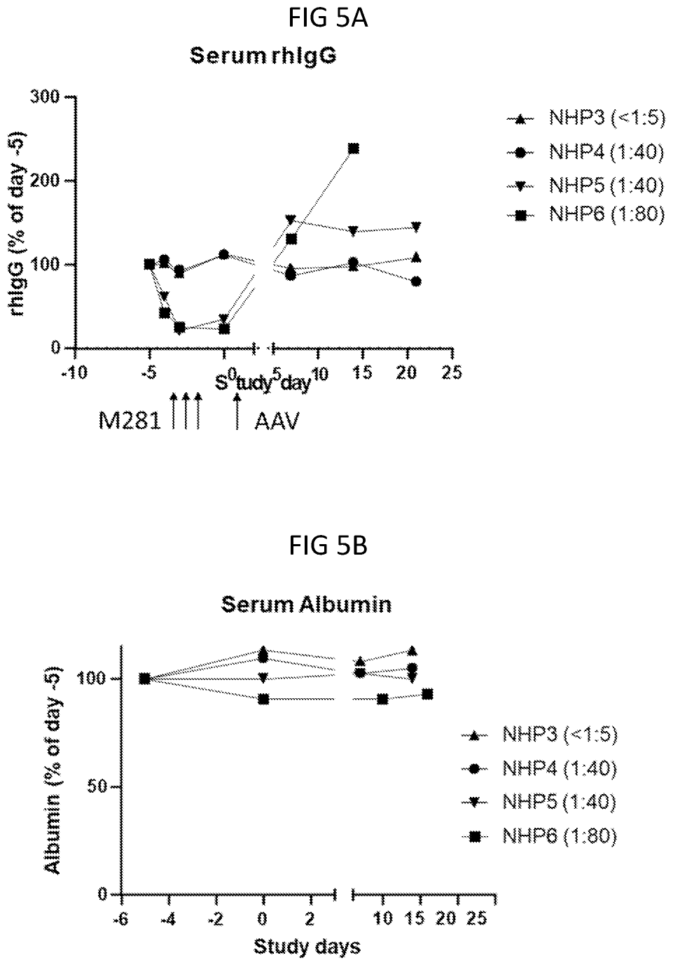

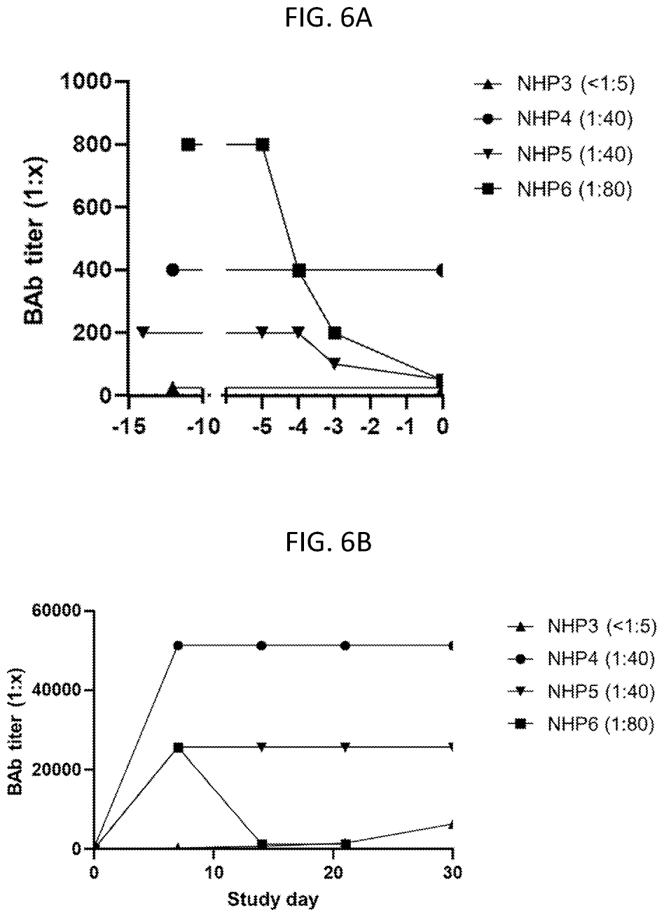

FIGS. 5A to 5B show M281 infusion reduced pre-existing NAb titer together with IgG in NHPs (Study 2). FIG. 5A shows levels of serum rhesus macaque IgG (rhIgG), plotted as percent of day −5, where administration of M281 (days −5, −4, and −3) and administration of AAV (day 0) are indicated by arrows on graph. FIG. 5B shows levels of serum albumin plotted as percent of day −5, wherein M281 administration is indicated by arrows on graph. FIGS. 6A to 6B show AAV-binding antibody titer (Study 2). FIG. 6A shows AAVhu68-non-neutralizing binding antibody (BAb) titer, during study Day −15 to Day 0, wherein administration of M281 (days −5, −4, and −3) and administration of AAV (day 0). FIG. 6B shows AAVhu68-non-neutralizing binding antibody (BAb) titer, during study Day 0 to Day 30.

AAVhu68-NAb titer is summarized in Table 4 below.

TABLE 4

| | M281 | BSL | Day −5 | Day −4 | Day −3 | Day 0 | Day 7 | Day 14 | Day 21 | Day 30 |
|---|---|---|---|---|---|---|---|---|---|---|
| NHP3 | − | <1:5 | | | | <1:5 | 1:160 | 1:640 | 1:640 | 1:640 |
| NHP4 | − | 1:40 | | | | 1:40 | 1:2560 | 1:1280 | 1:2560 | 1:1280 |
| NHP5 | + | 1:40 | 1:20 | 1:20 | <1:5 | 1:5 | 1:1280 | 1:1280 | 1:1280 | 1:1280 |
| NHP6 | + | 1:80 | 1:40 | 1:40 | 1:20 | 1:5 | 1:1280 | 1:640 | 1:320 | 1:320 |

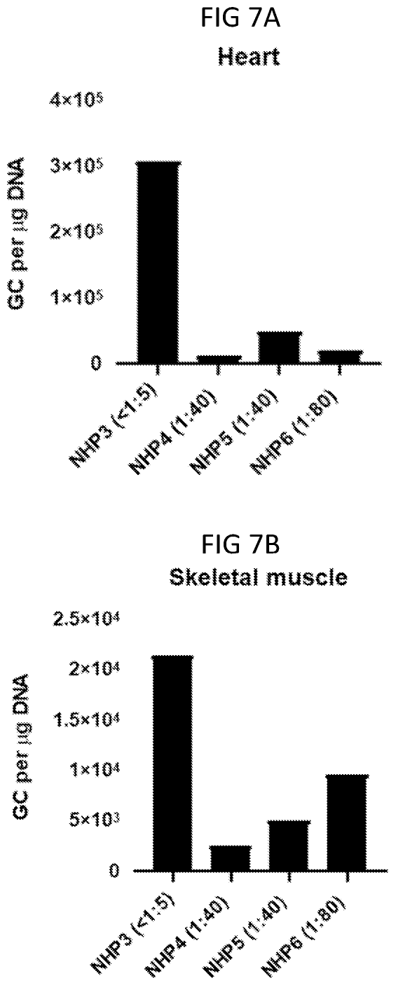

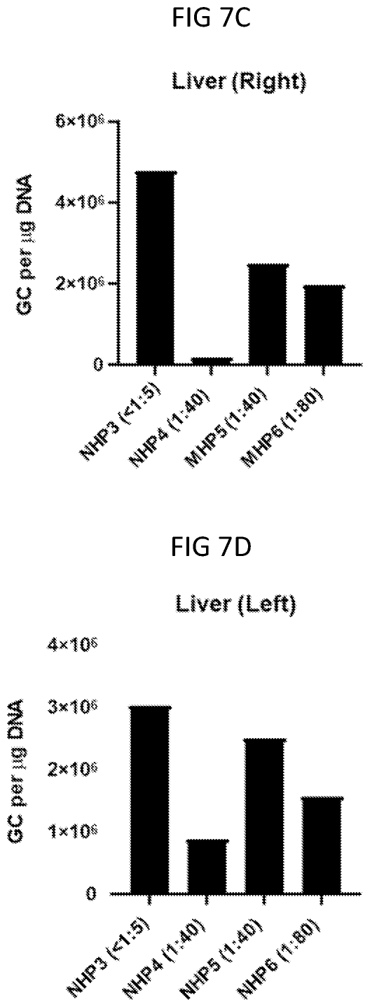

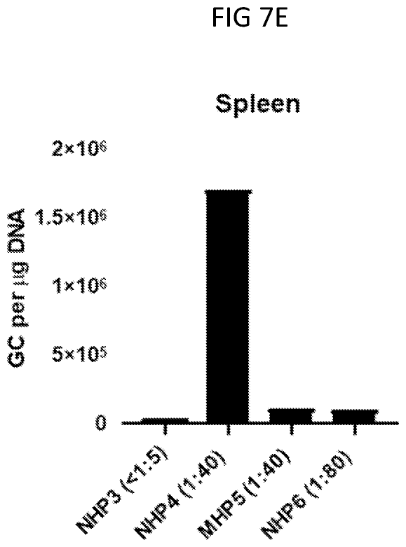

FIGS. 7A to 7E show vector genome biodistribution in various tissues harvested from Study 2, plotted as Genome Copy (GC) per micro-gram (μg) DNA. FIG. 7A shows vector genome biodistribution in heart. FIG. 7B shows vector genome biodistribution in skeletal muscle. FIG. 7C shows vector genome biodistribution in right lobe of liver. FIG. 7D shows vector genome biodistribution in left lobe of liver. FIG. 7E shows vector genome biodistribution in spleen.

In another study we will test the effect of pre-existing NAb on transduction efficacy after IV administration of AAV-TT3 (Test Transgene 3) in NHP, with or without FcRn blockade. Briefly, enrolled animals are divided into two groups based on the evaluated levels of pre-existing NAb and confirmed titer levels. Serum levels of NAb (neutralizing binding antibodies), baseline biomarkers, and transgene expression in plasma, heart, muscles and liver are measured as a readout of the study. TT2 in situ hybridization is expected to show improved expression of TT2 mRNA in the liver and heart from monkeys treated with M281.

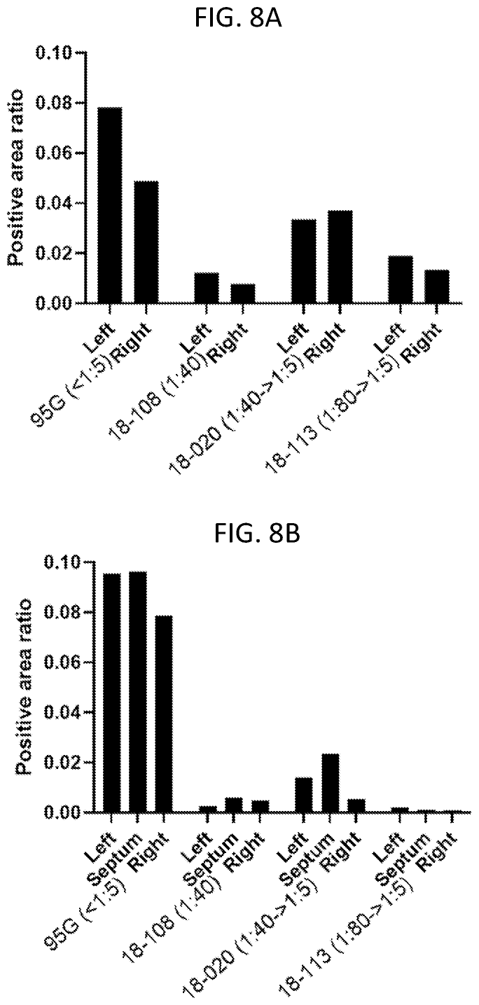

FIGS. 8A and 8B shows results of in situ hybridization examining TT2 mRNA expression levels in heart and liver tissues harvested from Study 2, plotted as positive area ratio. FIG. 8A shows results of in situ hybridization examining TT2 mRNA expression levels in liver tissue (left and right lobe) harvested from Study 2. FIG. 8B shows results of in situ hybridization examining TT2 mRNA expression levels in heart tissue (left, right ventricles and septum) harvested from Study 2.

### Example 3. M281 Antibody Production, Purification and Formulation for Intravenous Delivery

The example provided herein describe in vitro production of the immunoglobulin constructs.

Nucleic acid molecule encoding M281, comprising of M281-LC (light chain) and M281-HC (heavy chain), is obtained using standard techniques, i.e., Gene Synthesis Services by ThermoFisher Life Technologies. Further nucleic acid sequences encoding M281-LC or M281-HC are cloned into a suitable plasmid carrying vector genomes suitable for expression in a production host cell line. Plasmids carrying vector genomes are comprised of a CMV promoter (nucleotides 47 to 726 of SEQ ID NO: 1), a nucleic acid sequence encoding M281 constructs as described below, WRPE element (nucleotide 1514 to 2111 of SEQ ID NO: 1), and an thymidine kinase (TK) poly A signal (nucleotides 2115 to 2386 of SEQ ID NO: 1):
(a) M281-LC nucleic acid sequence (nucleotides 754 and 1467 of SEQ ID NO: 1) which encodes M281-LC protein of SEQ ID NO: 2, and/or
(b) M281-HC nucleic acid sequence (nucleotide 754 to 2154 of SEQ ID NO: 3) which encodes M281-HC protein of SEQ ID NO: 4.

Antibody constructs are cloned into a suitable plasmid are then used for expression in host cells, purified from production host cell line and formulated for intravenous delivery.

### Example 4. Anti-FcRN Antibody Treatment of Non-Human Primates with Pre-Existing Neutralizing Antibodies

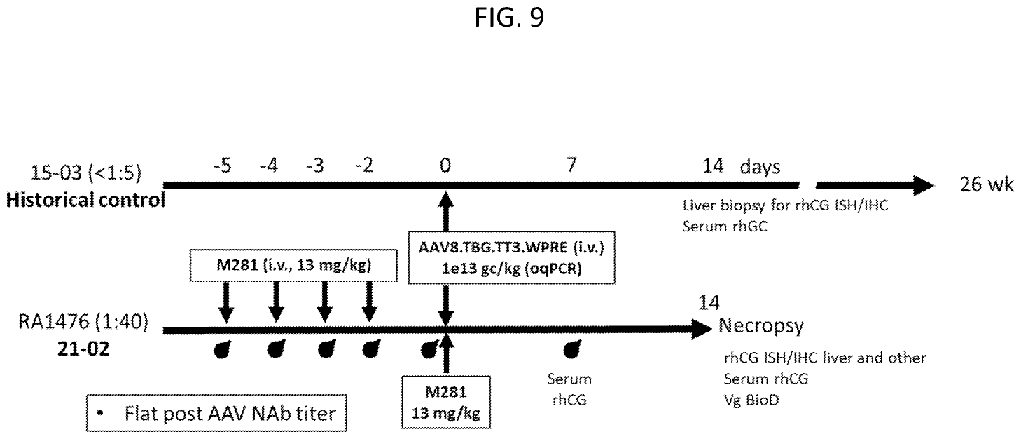

In this study, we tested the effect of M281 on pre-existing AAV8 NAb in NHPs using alternative M281 regimen. The NHPs were previously, in 2015, administered with AAV8 by intravenous administration. A historical AAV8 control was used for reference. The baseline AAV8 NAb levels were measured 1:40 for NHP study subject, and <1:5 for historical control. M281 was administered at a dose of up to 13 mg/kg. FIG. 9 shows a study design to evaluate the effect of blocking pre-existing FcRn NAb titer following re-administration of AAV8.TT3 (test transgene 3) at a dose of 1×10¹³ GC/kg. At day 14 serum levels of TT3 expression were measured. At day 14 necropsy was performed on NHP study subject, and liver biopsy was performed on historical control subject. The collected tissue was used for analysis of TT3 expression (ISH/IHC). Tissue and samples collected from study subject were also analyzed for serum levels of TT3 expression and vector biodistribution.

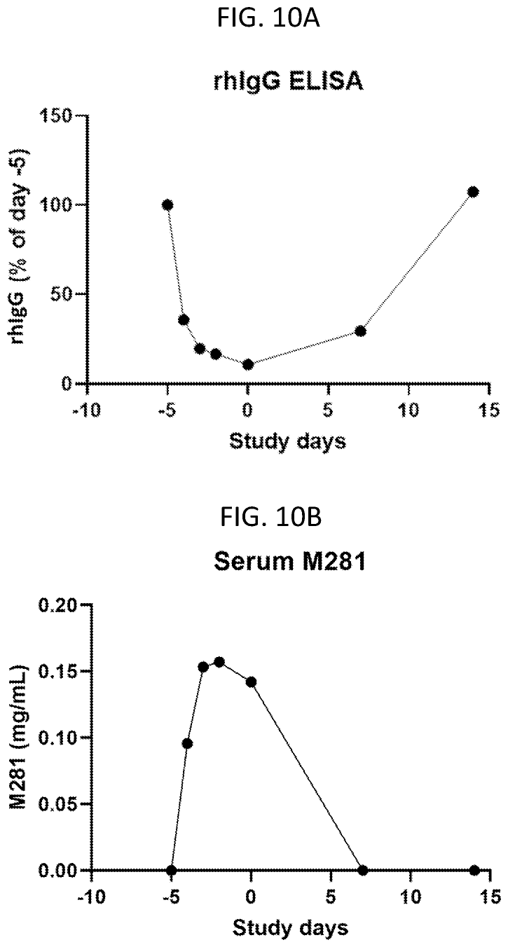

FIGS. 10A and 10B show results of AAV8.TT3 re-administration study, in which M281 administration reduced pre-existing NAb titer (AAV8) together with IgG in NHP (previously administered AAV8.TT3). FIG. 10A shows serum levels of rhesus macaque IgG (rhIgG), plotted as percent of day −5, where NHP was administered M281 at days −5, −4, −3, and −2 and AAV8.TT3 at day 0. FIG. 10B shows measured serum levels of M281 plotted as mg/mL.

Table 5 below shows summary of the AAV8 NAb levels as examined above. Table 6 below shows summary of results of AAV8 binding ELISA assay.

TABLE 5

| | | AAV8 NAb in HEK293 cells | | | | |
|---|---|---|---|---|---|---|
| Animal | | Rep | Rep | Rep | Rep | Average |
| ID | Study Day | 1 | 2 | 3 | 4 | of RLU/s |
| RA1476 | Pre-Screen | 40 | N/A | N/A | N/A | 40 |
| | Baseline | 80 | 40 | 40 | 40 | 80 |
| | Day −5 | 40 | 40 | 40 | 40 | 40 |
| | Day −4 | 80 | 40 | 40 | 40 | 40 |
| | Day −3 | 40 | 40 | 40 | 20 | 40 |
| | Day −2 | 40 | 20 | 20 | 40 | 40 |
| | Day 0 | 10 | 10 | 10 | 10 | 10 |

TABLE 6

| | | AAV8 Binding Elisa | |
|---|---|---|---|
| Animal ID | Study Day | Rep 1 | Rep 2 |
| RA1476 | Pre-Screen | N/A | N/A |
| | Baseline | 400 | 400 |
| | Day −5 | 400 | 400 |
| | Day −4 | 200 | 200 |
| | Day −3 | 200 | 200 |
| | Day −2 | 200 | 200 |
| | Day 0 | 100 | 100 |

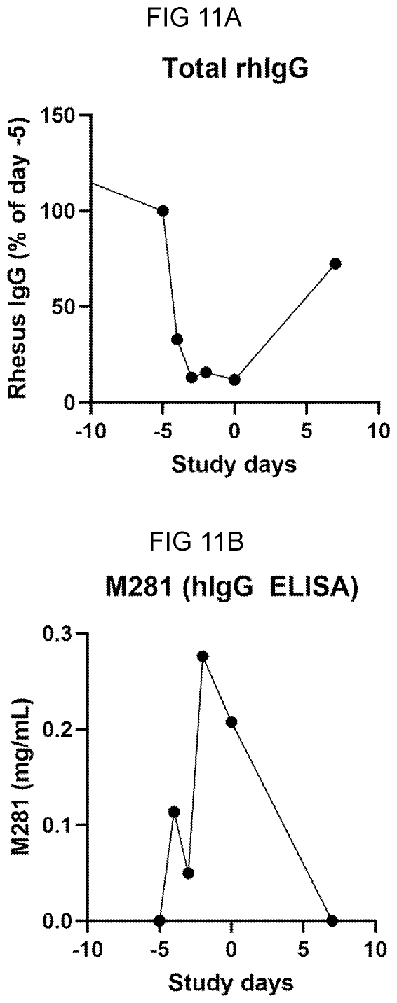

FIGS. 11A and 11B shows results of another AAV8.TT3 study, in which M281 administration reduced pre-existing NAb titer (AAV8) together with IgG in NHP with pre-existing NAb+(1:20) by natural infection. FIG. 11A shows total rhesus macaque IgG levels (rhIgG) plotted as percent of day −5, where NHP was administered M281 at days −5, −4, −3, and −2 and AAV8.TT3 at day 0. FIG. 11B shows serum M281 levels (hIgG) plotted as mg/mL and measured using ELISA. Table 7 shows summary of the AAV8 NAb levels as examined above. Table 8 below shows summary of results of AAV8 binding ELISA assay.

TABLE 7

| | | AAV8 NAb in HEK293 cells | | | | |
|---|---|---|---|---|---|---|
| Animal | | Rep | Rep | Rep | Rep | Average |
| ID | Study Day | 1 | 2 | 3 | 4 | of RLU/s |
| 181262 | Pre-Screen | 10 | 20 | N/A | N/A | 20 |
| | Baseline | 10 | 20 | 20 | 10 | 20 |
| | Day −5 | 10 | 10 | 10 | 10 | 10 |
| | Day −4 | 5 | 10 | 5 | 5 | 5 |
| | Day −3 | 10 | 10 | 10 | 10 | 10 |
| | Day −2 | <5 | <5 | <5 | <5 | <5 |
| | Day 0 | | | | | <5 |

TABLE 8

| | | AAV8 Binding Elisa | |
|---|---|---|---|
| Animal ID | Study Day | Rep 1 | Rep 2 |
| 181262 | Pre-Screen | N/A | N/A |
| | Baseline | N/A | N/A |
| | Day −5 | 20 | 20 |
| | Day −4 | 20 | 20 |
| | Day −3 | 10 | 10 |
| | Day −2 | <10 | <10 |

TABLE
(Sequence Listing Free Text)
The following information is provided for sequences
containing free text under numeric identifier <223>.

| SEQ ID NO: | |
|---|---|
| (containing free | |
| text) | Free text under <223> |
| 1 | <220> |
| | <223> M281-LC expression plasmid |
| | <220> |
| | <221> promoter |
| | <222> (47) . . . (726) |
| | <223> CMV Promoter |
| | <220> |
| | <221> CDS |
| | <222> (754) . . . (1467) |
| | <223> M281_LC |
| | <220> |
| | <221> misc_feature |
| | <222> (1514) . . . (2111) |
| | <223> WRPE |
| | <220> |
| | <221> misc_feature |
| | <222> (2115) . . . (2386) |
| | <223> TK pA signal |
| | <220> |
| | <221> promoter |
| | <222> (2855) . . . (3224) |
| | <223> SV40 early promoter |
| | <220> |
| | <221> misc_feature |
| | <222> (3260) . . . (4054) |
| | <223> Neo-R |
| | <220> |
| | <221> polyA_signal |
| | <222> (4230) . . . (4360) |
| | <223> SV40 pA signal |
| | <220> |
| | <221> rep_origin |
| | <222> (4743) . . . (5416) |
| | <223> pUC ori |
| | <220> |
| | <221> misc_feature |
| | <222> (5561) . . . (6421) |
| | <223> Amp-R |
| | <220> |
| | <221> promoter |
| | <222> (6422) . . . (6520) |
| | <223> bla promoter |
| 2 | <220> |
| | <223> Synthetic Construct |
| 3 | <220> |
| | <223> M281-HC expression plasmid |
| | <220> |
| | <221> promoter |
| | <222> (47) . . . (726) |
| | <223> CMV promoter |
| | <220> |
| | <221> CDS |
| | <222> (754) . . . (2154) |
| | <223> M281_HC |
| | <220> |
| | <221> misc_feature |
| | <222> (2201) . . . (2798) |
| | <223> WRPE |
| | <220> |
| | <221> misc_feature |
| | <222> (2802) . . . (3073) |
| | <223> TK pA signal |
| | <220> |
| | <221> promoter |
| | <222> (3542) . . . (3911) |
| | <223> SV40 ealry promoter |
| | <220> |
| | <221> misc_feature |
| | <222> (3947) . . . (4741) |
| | <223> Neo-R |
| | <220> |
| | <221> polyA_signal |
| | <222> (4917) . . . (5047) |
| | <223> SV40 pA signal |
| | <220> |
| | <221> rep_origin |
| | <222> (5430) . . . (6103) |
| | <223> pUC ori |
| | <220> |
| | <221> misc_feature |
| | <222> (6248) . . . (7108) |
| | <223> Amp-R |
| | <220> |
| | <221> promoter |
| | <222> (7109) . . . (7207) |
| | <223> bla promoter |
| 4 | <220> |
| | <223> Synthetic Construct |
| 5 | <220> |
| | <223> M281_LC nucleic acid sequence |
| 6 | <220> |
| | <223> M281_HC nucleic acid sequence |
| 7 | <220> |
| | <223> M281_LC amino acid sequence |
| 8 | <220> |
| | <223> M281_HC amino acid sequence |
| 9 | <220> |
| | <223> CDR-L1 nucleic acid sequence |
| 10 | <220> |
| | <223> CDR-L1 amino acid sequence |
| 11 | <220> |
| | <223> CDR-L2 nucleic acid sequence |
| 12 | <220> |
| | <223> CDR-L2 amino acid sequence |
| 13 | <220> |
| | <223> CDR-L3 nucleic acid sequence |
| 14 | <220> |
| | <223> CDR-L3 amino acid sequence |
| 15 | <220> |
| | <223> CDR-H1 nucleic acid sequence |
| 16 | <220> |
| | <223> CDR-H1 amino acid sequence |
| 17 | <220> |
| | <223> CDR-H2 nucleic acid sequence |
| 18 | <220> |
| | <223> CDR-H2 amino acid sequence |
| 19 | <220> |
| | <223> CDR-H3 nucleic acid sequence |
| 20 | <220> |
| | <223> CDR-H3 amino acid sequence |
| 21 | <220> |
| | <223> CDR-H1 variant 1 |
| 22 | <220> |
| | <223> CDR-H1 variant 2 |
| 23 | <220> |
| | <223> CDR-H2 variant 1 |
| 24 | <220> |
| | <223> CDR-H2 variant 2 |
| 25 | <220> |
| | <223> CDR-H2 variant 3 |
| 26 | <220> |
| | <223> Z_FcRn-2 |
| 27 | <220> |
| | <223> Z_FcRn-4 |
| 28 | <220> |
| | <223> Z_FcRn-16 |
| 29 | <220> |
| | <223> SYN746 |
| 30 | <220> |
| | <223> SYN1327 |
| 31 | <220> |
| | <223> SYN1327-modified |
| | <220> |
| | <221> MOD_RES |
| | <222> (3) . . . (3) |
| | <223> Pen |
| | <220> |
| | <221> MOD_RES |
| | <222> (9) . . . (9) |
| | <223> Sar |
| | <220> |
| | <221> MOD_RES |
| | <222> (10) . . . (10) |
| | <223> NMeLeu |
| 32 | <220> |
| | <223> 98420-1 amino acid sequence |
| 33 | <220> |
| | <223> 98420-2 |
| 34 | <220> |
| | <223> 98420-3 |
| 35 | <220> |
| | <223> 98420-4 |
| 36 | <220> |
| | <223> 98420-5 |
| 37 | <220> |
| | <223> DX2504LC |
| 38 | <220> |
| | <223> DX2504HC |
| 39 | <220> |
| | <223> DX2507LC |
| 40 | <220> |
| | <223> DX2507HC |
| 41 | <220> |
| | <223> DX-CDR-L3 |
| 42 | <220> |
| | <223> DX-CDR-L2 |
| 43 | <220> |
| | <223> DX-CDR-H1 |
| 44 | <220> |
| | <223> DX-CDR-H2 |
| 45 | <220> |
| | <223> hFcRN amino acid sequence |

All publications cited in this specification are incorporated herein by reference in their entireties. U.S. Provisional Patent Application No. 63/040,381, filed Jun. 17, 2020, U.S. Provisional Patent Application No. 63/135,998, filed Jan. 11, 2021, and U.S. Provisional Patent Application No. 63/152,085, filed Feb. 22, 2021 are incorporated herein by reference. The appended Sequence Listing labeled “UPN-20-9394PCT_ST25” is incorporated by reference. While the invention has been described with reference to particular embodiments, it will be appreciated that modifications can be made without departing from the spirit of the invention. Such modifications are intended to fall within the scope of the appended claims.

1. A combination regimen for treating a patient with immunoglobulin G (IgG) neutralizing antibodies to a selected AAV capsid or an AAV capsid serologically cross-reactive to the selected capsid, the regimen comprising (a) administering an AAV viral vector comprising the selected AAV capsid and a vector genome comprising a nucleic acid sequence encoding a gene product operably linked to regulatory sequences which direct expression thereof in a target cell, and (b) co-administering a ligand which specifically prevents binding between human neonatal Fc receptor (FcRn) and the neutralizing antibodies without interfering with albumin binding to FcRn.

2. The combination regimen according to claim 1, wherein the ligand is selected from a peptide, protein, an RNAi sequence, or a small molecule.

3. The combination regimen according to claim 1, wherein the ligand is a monoclonal antibody, an immunoadhesin, a camelid antibody, a Fab fragment, an Fv fragment, or an scFv fragment directed against FcRn.

4. The combination regimen according to claim 1, wherein the ligand is a monoclonal antibody selected from nipocalimab (M281), rozanolixizumab; IMVT-1401, RVT-1401, HL161, HBM916, efgartigimod, orilanolimab (SYNT001), SYNT002, ABY-039, DX-2507, derivatives or combinations thereof.

5. The combination regimen according to claim 1, wherein the ligand is nipocalimab (M281) or an immunoglobulin construct which comprises three or more CDRs thereof selected from:
(a) heavy chain CDRs of (i) CDR H1, SEQ ID NO: 16 or a sequence at least 99% identical thereto, (ii) CDR H2, SEQ ID NO:18 or a sequence at least 99% identical thereto, and (iii) CDR H3, SEQ ID NO: 20 or a sequence at least 99% identical thereto; or
(b) light chain CDRs of: CDR L1, SEQ ID NO: 10 or a sequence at least 99% identical thereto, CDR L2, SEQ ID NO:12 or a sequence at least 99% identical thereto, CDR L3, SEQ ID NO: 14, or a sequence at least 99% identical thereto.

6. The combination regimen according to claim 5, wherein nipocalimab or the immunoglobulin construct comprises:
(a) heavy chain CDRs of (i) CDR H1, SEQ ID NO: 16, (ii) CDR H2, SEQ ID NO:18, and (iii) CDR H3, SEQ ID NO: 20; or
(b) light chain CDRs of: CDR L1, SEQ ID NO: 10, CDR L2, SEQ ID NO:12, CDR L3, SEQ ID NO: 14.

7. The combination regimen according to claim 1, wherein the ligand is expressed in vivo following delivery of a vector comprising sequences encoding the ligand operably linked to regulatory sequences which direct expression of the ligand.

8. The combination regimen according to claim 1, wherein the rAAV encoding the gene product has a capsid selected from AAV1, AAV2, AAV3, AAV5, AAV7, AAV8, AAV9, AAVrh10, AAVrh91, AAVhu37, or AAVhu68.

9. The combination regimen according to claim 1, wherein prior to delivery of the combination regimen, the patient has a neutralizing titer greater than 1:5 against the rAAV capsid or a serologically cross-reactive capsid as determined in an in vitro assay.

10. The combination regimen according to claim 1, wherein the ligand is delivered one to seven days prior to delivery of the rAAV.

11. The combination regimen according to claim 1, wherein the ligand is delivered daily.

12. The combination regimen according to claim 1, wherein the ligand is delivered on the same day as when the rAAV is delivered.

13. The combination regimen according to claim 1, wherein the ligand is delivered for one day to four weeks and/or for four weeks to 6 months post-rAAV delivery.

14. The combination regimen according to claim 1, wherein the ligand is delivered orally.

15. The combination regimen according to claim 1, wherein the rAAV is delivered systemically, optionally via intraperitoneal, intravenous, intramuscular, or intranasally, or intrathecally.

16. The combination regimen according to claim 1, wherein the patient has not previously received gene therapy treatment or gene delivery using an AAV prior to delivery of the viral vector in combination with the inhibitory ligand such that the patient's pre-existing neutralizing antibodies are a result of wild-type viral vector infection.

17. The combination regimen according to claim 1, wherein the regimen further comprises co-administering one or more of: (a) a steroid or combination of steroids; and/or (b) an IgG-cleaving enzyme; and (c) an inhibitor of Fc-IgE binding; (d) an inhibitor of Fc-IgM binding; (e) an inhibitor of Fc-IgA binding; and/or (f) gamma interferon.

18.-19. (canceled)

20. A method for increasing the patient population for which rAAV-mediated gene therapy is effective,
said method comprising co-administering to a patient from a population having a neutralizing antibody titer to a selected AAV viral capsid or a serologically cross-reactive capsid which prevents effective transfer and expression levels of the transgene product:
(a) a recombinant rAAV having a selected AAV capsid and a vector genome packaged in the selected capsid; and
(b) a ligand which specifically binds a neonatal Fc receptor (FcRn) prior to delivery of the gene therapy vector without substantially interfering with FcRn-albumin binding,
wherein the ligand blocks binding of the FcRN to immunoglobulin G (IgG), and permits effective amounts of the gene therapy product to be expressed in the patient.

21.-32. (canceled)

33. The method according to claim 20, wherein the immunoglobulin construct is delivered via a different route of than the rAAV is delivered.

34. The method according to claim 20, wherein the immunoglobulin construct is delivered orally.

35. The method according to claim 20, wherein the patient was predetermined to have a neutralizing titer greater than 1:5 as determined in an in vitro assay.

36. The method according to claim 20, wherein the method is part of a regimen which further comprises co-administering one or more of: (a) a steroid or combination of steroids and/or (b) an IgG-cleaving enzyme, (c) an inhibitor of Fc-IgE binding; (d) an inhibitor of Fc-IgM binding; (e) an inhibitor of Fc-IgA binding; and/or (f) gamma interferon.
